(19) United States
(12) Patent Application Publication
(10) Pub. No.: US 2020/0330230 A1
(43) Pub. Date: Oct. 22, 2020

(54) SPINAL INSTRUMENTATION TO ENHANCE OSTEOGENESIS AND FUSION

(71) Applicant: OSTEOVANTAGE, INC., Fayetteville, AR (US)

(72) Inventors: Matthew R. MACEWAN, St. Louis, MO (US); Eric C. LEUTHARDT, St. Louis, MO (US); Daniel W. MORAN, Ballwin, MO (US)

(21) Appl. No.: 16/093,034

(22) PCT Filed: Apr. 11, 2017
(86) PCT No.: PCT/US2017/027052

§ 371 Date: Oct. 11, 2018

Related U.S. Application Data
(60) Provisional application No. 62/321,092, filed on Apr. 11, 2016.

(51) Int. Cl. A61F 2/28; A61B 17/70; A61B 17/86; A61N 1/20

(52) U.S. Cl. CPC A61F 2/28; A61B 17/7002; A61B 17/7032; A61B 2017/564; A61B 17/8625; A61N 1/205; A61B 17/8615

(57) ABSTRACT

Systems and methods for producing osteogenic effect in spinal fixation systems using selectively anodized components are described. Anodization patterns can be selected to produce a desired electric field and osteogenic effect in tissues and structures surrounding the selectively anodized component.

### CROSS REFERENCE TO RELATED APPLICATIONS

This application claims the benefit of U.S. Provisional Application No. 62/321,092, filed Apr. 11, 2016.

### INCORPORATION BY REFERENCE

All publications and patent applications mentioned in this specification are incorporated herein by reference in their entirety to the same extent as if each individual publication or patent application was specifically and individually indicated to be incorporated by reference.

### FIELD

The application relates to the fields of spinal fixation and osteogenesis.

### BACKGROUND

Bone growth is desirable in many instances, such as when vertebrae in a patient's spine are fused to overcome pain and other effects caused by inter-vertebral movement or intra-vertebral movement. Although bone growth occurs naturally, it can be stunted or stopped by various factors such as tobacco, alcohol and steroid usage, poor bone stock, and age. Moreover, stimulating bone growth to speed recovery is desirable in some instances such as when an injured athlete wishes to return to her sport quickly. Thus, there is a need for stimulating bone growth in individuals.

Bone growth can be stimulated by various means. One such means for stimulating bone growth is by passing an electrical current through the bone. When fusing vertebrae in a patient's spine, various means have been used to stimulate bone growth. For example, some stimulators include wire electrodes embedded in bone fragments grafted to a region of the patient's back containing the vertebrae to be fused. Direct electrical current is applied to the electrodes to stimulate bone growth and fuse the fragments and adjoining vertebrae. To permit the current to be applied for extended periods of time while permitting the patient to be mobile, a generator is connected to the wire electrodes and implanted between the skin and muscle near the patient's vertebral column. The generator provides a continuous low amperage direct current (e.g., 40 μA) for an extended period of time (e.g., six months). After the vertebrae are fused, the generator and leads are surgically removed. Although these embedded electrodes are generally effective, the wire electrodes are susceptible to failure, requiring additional surgery to repair them. Moreover, placement of the wire electrodes is less than precise, allowing some of the current to pass through undesirable areas of tissue and encouraging bone to form where it is unneeded. Imprecise delivery of direct current could also potentially have adverse effects. Further, imprecise placement may require more energy to be provided to the electrodes than otherwise necessary to be optimally effective. Thus, there are several drawbacks and potential problems associated with devices such as these.

Although small amounts of bone movement can stimulate growth, it is generally desirable to limit movement between the bones or bone fragments being fused. There are several known means for limiting bone movement. Among these means for limiting bone movement are plates, rods and screws. The plates and rods are typically held in position by screws which are mounted in the bone or bones being fused. FIG. 1 illustrates a spinal fixation system 100. The system comprises screws 101, 102 with screw bodies 104, 105 implanted into a vertebra 103 to immobilize the vertebra. The screws 101, 102 are seated within tulips 106, 107 which receive rods 108, 109. Screws 101, 102 are used to fix rods within the tulips 106, 107. As previously mentioned, the screws 10 are used for attaching rods 14 and/or plates (not shown) to vertebrae to hold the vertebrae in position while healing and fusion occurs.

Leuthardt (U.S. Pat. No. 8,784,411) describes the use of screws for pedicle fixation and precise delivery of energy and current to the fixated bone and proximal anatomical regions or features. Leuthardt discloses a screw with an electrically conductive and an electrically insulated portion which serves as a conduit to deliver direct current to a specific portion of the instrumented bone. Sloan (U.S. Patent Publication No. 2015/0088203) and Berger (U.S. Pat. No. 8,380,319) also describe variations to the rigid instrument design to allow existing instruments (e.g., those shown in FIG. 1) to be modified to provide the power, control, circuitry, telemetry, etc. for spinal system simulators. While such systems exist, there is still a need for improvements in the component designs and implementation for improved bone growth outcomes.

### SUMMARY OF THE DISCLOSURE

In one aspect a system for spinal fixation and osteogenesis is provided. The system comprises a pedicle screw comprising a selectively anodized surface configured to generate a desired electric field when energized; a power source; an electrical connector connecting the power source and pedicle screw and configured to provide a constant level of direct current to the pedicle screw; and a saddle configured to receive the pedicle screw and comprising a notch configured to allow passage of the electrical connector from the screw to external components.

In another aspect, a system for spinal fixation and osteogenesis is provided. The system comprises a power source; a tulip comprising a channel; a rod configured to be positioned within the channel; and a pedicle screw; a saddle comprising a notch along a bottom surface shaped to mate with a top of the pedicle screw, the saddle configured to be positioned between the tulip and the rod, wherein at least one of the tulip, rod, screw seat, and pedicle screw comprises a selectively anodized surface configured to generate a desired electric field when energized using a constant current supplied by the power source, and wherein at least one of the tulip.

In some embodiments, the power source comprises a hermetically sealed titanium enclosure. In some embodiments, the enclosure comprises a battery. In some embodiments, the power source is configured to produce direct current of about 10-100 μA. In some embodiments, the system comprises a wireless communication module and/or electrical circuitry. The system can comprise an electrical connector configured to connect the power source to the component comprising the selectively anodized surface. In some embodiments, the connector comprises an insulated micro-wire lead. In some embodiments, the pedicle screw comprises the selectively anodized surface. The connector can be attached to the pedicle screw at a head of the screw and the notch in the saddle permits passage of the connector. The selectively anodized surface can comprise a layer positioned at a top portion of the screw. The layer can extend over at least a portion of a head and shaft of the screw. In some embodiments, the selectively anodized surface extends over about 90% of a total length of the screw. In some embodiments, the selectively anodized surface comprises an anodized portion and an unanodized portion. In some embodiments, the anodized portion is configured to prohibit delivery of current to adjacent tissue when the system is implanted. The unanodized portion can be configured to support delivery of current to adjacent tissue when the system is implanted. In some embodiments, the selectively anodized surface is configured to selectively direct electrical stimulation to the vertebral body and intervertebral disc space without directing electrical stimulation to the spinal canal. The selectively anodized surface can comprise a single and/or a variable thickness. The variable thickness can be linearly and/or exponentially graded. In some embodiments, the selectively anodized surface comprises a first region of a consistent thickness anodization and a second region of a variable thickness anodization. The first region can comprise about 25% a length of the component. The second region can comprise about 75% a length of the component. In some embodiments, the selectively anodized surface comprises a segmented coating comprising two or more discontinuous regions of anodization. The first region can be positioned at a top portion of the screw. The second region can be positioned at the bottom portion of the screw. The first region can comprise about 60% a length of the screw. The second region can comprise about 10% a length of the screw. In some embodiments, an unanodized region comprising about 30% a length of the screw is positioned between the first region and the second region. The screw can have a length of about 35 mm. In some embodiments, the anodized surface is created with a driving voltage of greater than 80V. In some embodiments, the anodized surface comprises Type I anodization.

In another aspect, a spinal fixation system is provided. The system comprises a first selectively anodized pedicle screw configured to be implanted at a first vertebral level; a second selectively anodized pedicle screw configured to be implanted at a second vertebral level, different from the first level, wherein the first and second screws are configured to deliver a desired electric field to surrounding tissues and structures when energized; and a power source configured to deliver constant current to the first and second screws.

In some embodiments, the first and second screws have the same anodization pattern. The first and second screws can have different anodization patterns. In some embodiments, the first and second screws are configured to function independent of one another to induce osteogenic effect in tissue directly adjacent to each screw when the screws are energized. In some embodiments, the first and second screws are configured to work in combination to produce a synergistic electric field when the screws are energized. In some embodiments, at least one of the screws comprises an anodization layer positioned at a top portion of the screw.

The layer can extend over at least a portion of a head and shaft of the screw. In some embodiments, at least one of the screws comprises a selectively anodized surface that extends over about 90% of a total length of the screw. In some embodiments, at least one of the screws comprises a selectively anodized surface that comprises an anodized portion and an unanodized portion. In some embodiments, the anodized portion is configured to prohibit delivery of current to adjacent tissue when the system is implanted. The unanodized portion can be configured to support delivery of current to adjacent tissue when the system is implanted. In some embodiments, at least one of the screws comprises a selectively anodized surface that is configured to selectively direct electrical stimulation to the vertebral body and intervertebral disc space without directing electrical stimulation to the spinal canal. At least one of the screws can comprise a selectively anodized surface that can a single and/or a variable thickness. The variable thickness can be linearly and/or exponentially graded. In some embodiments, at least one of the screws comprises a selectively anodized surface that comprises a first region of a consistent thickness anodization and a second region of a variable thickness anodization. The first region can comprise about 25% a length of the component. The second region can comprise about 75% a length of the component. In some embodiments, at least one of the screws comprises a selectively anodized surface that comprises a segmented coating comprising two or more discontinuous regions of anodization. The first region can be positioned at a top portion of the screw. The second region can be positioned at the bottom portion of the screw. The first region can comprise about 60% a length of the screw. The second region can comprise about 10% a length of the screw. In some embodiments, at least one of the screws comprises an unanodized region comprising about 30% a length of the screw is positioned between the first region and the second region. In some embodiments, a field created in a region distant to the first screw is different from a field created in a region distant to the second screw. The system can further comprise a third selectively anodized pedicle screw configured to be implanted at a third vertebral level, different from the first and second levels, such that the second pedicle screw is positioned between the first and third pedicle screws. In some embodiments, the third screw has a same anodization pattern as the first and second screws. The third screw can have a different anodization pattern as the first and second screws. In some embodiments, the second and third screws are configured to function independent of one another to induce osteogenic effect in tissue directly adjacent to each screw when the screws are energized. In some embodiments, the second and third screws are configured to work in combination to produce a synergistic electric field when the screws are energized.

In another aspect, a method for inducing osteogenic effect is provided. The method comprises selecting an appropriate anodization pattern for a selectively anodized pedicle screw; implanting a spinal fixation system comprising the selectively anodized pedicle screw; energizing the pedicle screw using a constant level of direct current, thereby producing a desired electrical field in an area proximate to the pedicle screw; and producing an osteogenic effect in surrounding tissue and structures. Energizing the screw can comprise applying a direct current of about 60 μA. The method can comprise connecting the screw to a power source. The method can further comprise implanting a second selectively anodized pedicle screw. The method can further comprise implanting a third selectively anodized pedicle screw.

In another aspect, a system for spinal fixation and osteogenesis is provided. The system comprises a pedicle screw comprising an electrical connector extending from a head of the screw; a saddle shaped to receive a head of the pedicle screw and comprising a notch configured to allow passage of the electrical connector therethrough; a tulip configured shaped to receive the saddle; and a rod shaped to be positioned above the saddle and within a channel of the tulip. In some embodiments, the screw comprises a selectively anodized surface configured to generate a desired electric field when energized using a constant current. In some embodiments, the screw comprises a selectively anodized pattern as described herein. In some embodiments, the tulip comprises a notch configured to allow passage of the connector therethrough. In some embodiments, the channel of the tulip exposes the notch of the saddle. In some embodiments, the system comprises a driver configured to engage the head of the screw and a slot on a side of the driver to allow passage of the connector therethrough. The screw head can comprise an aperture for receiving the connector. The aperture can be positioned within a vestibule. The vestibule can be filled with a sealant around an attachment point of the connector and the screw. The aperture can be positioned within a receptacle in the screw head for engaging a driver. The point at which the connector attaches to the screw can be insulated. A top portion of the screw head and the screw can be insulated. A portion of the screw at which the screw connects to the connector can be uninsulated.

In another aspect, embodiments of a pedicle screw are provided. The pedicle screw comprises a head comprising a receptacle shaped to mate with a driver head; a connector aperture positioned within the receptacle; a connector attachment configured for attaching the connector to the connector aperture; and a vestibule surrounding the connector aperture.

The pedicle screw can comprise a connector positioned within the connector aperture. In some embodiments, the pedicle screw comprises sealant positioned within the vestibule and around the connector. In some embodiments, the pedicle screw comprises a channel in a side wall of the pedicle screw allowing access to the vestibule. In some embodiments, the pedicle screw comprises a selectively anodized surface as described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a horizontal cross section of a conventional electrically conductive screw installed in a vertebra;

FIG. 2A is a side elevation of an embodiment of a screw of the present invention and illustrates a cross sectional view of an exemplary fixation system comprising a conductive screw with anodized surface coating;

FIG. 2B illustrates a cross sectional view of an embodiment of a fixation system comprising a conductive screw with single thickness anodized surface coating

FIG. 2C illustrates a cross sectional view of an embodiment of a fixation system comprising a conductive screw with varying thickness anodized surface coating

FIG. 2D illustrates a cross sectional view of an embodiment of a fixation system comprising a conductive screw with segmented anodized surface coating

FIG. 3 is a side elevation of a portion of a spine with an embodiment of a two-level fixation system installed therein, the system comprising multiple conductive and selectively anodized screws connected via leads to a power supply.

FIG. 4 is a side elevation of a portion of a spine with an embodiment of a multi-level (e.g. three) level fixation system installed therein, the system comprising multiple conductive and selectively anodized screws connected via leads to a power supply

FIG. 5 illustrates an embodiment of an integrated power supply and attachment device for attaching an integrated power supply to a fixation system comprising multiple conductive and selectively anodized screws

FIG. 6 illustrates an embodiment of an integrated power supply contained within or attached to the head of multiple conductive and selectively anodized screws within a fixation system.

FIG. 7 illustrates an embodiment of an integrated power supply contained within a screw cap or set screw and connected to multiple conductive and selectively anodized screws within a fixation system.

FIG. 8 demonstrates electric field distributions resulting from electrical activation of pedicle screws modified with graded anodization patterns extending over either 100% or 50% of the length of the screw body.

FIG. 9 demonstrates electric field distributions resulting electrical activation of a one-level spinal fixation system.

FIG. 10 demonstrates electric field distributions resulting electrical activation of a one-level spinal fixation system comprising screws with differing anodization patterns.

FIGS. 11A-11B illustrate an embodiment of a connection between an electrical connector and a screw.

FIGS. 12A-12C illustrate an embodiment of a spinal system accommodating a connection between an electrical connector and a screw.

FIG. 13 illustrates an embodiment of a connection between an electrical connector and a screw.

FIGS. 14A-F illustrate a coronal view of a 3D reconstruction of the fusion mass at the L4-5 disc space.

FIGS. 15A-F illustrate a sagittal view of a 3D reconstruction of the fusion mass at the L4-5 disc space.

FIGS. 16A-F illustrate a coronal view of radiographic examination of bony fusion at the L4-5 disc space.

FIGS. 17A-F illustrate a sagittal view of radiographic examination of bony fusion at the L4-5 disc space.

FIGS. 18A-D illustrate quantitative analysis of trabecular continuity across the L4-5 disc space using sagittal micro-CT scans.

FIGS. 19A-C illustrate quantitative analysis of coronal micro-CT scans showing bone density surrounding pedicle screw beds.

Corresponding reference characters indicate corresponding parts throughout the several views of the drawings.

### DETAILED DESCRIPTION

Disclosed herein is a system for pedicle fixation that provides rigid fixation of the vertebrae while also generating and delivering a desired direct current and electric field for promoting osteogenic bone growth. The desired electrical stimulus and field can be formed by using energized components (e.g., screws, tulips, screw caps, rods) with particular anodization patterns. In some embodiments, a single energized component (e.g., a screw) can be used to form a desired field. In some embodiments, multiple energized components (e.g. two or more screws) working together can be used to form a desired field. In some embodiments, multiple types of energized components (e.g. a screw and a rod) working together can be used to form a desired field. For example, in a two or three level fixation system, the effects of energized components positioned at various anatomical locations or levels can combine to produce a desired result.

As described above, the prior art teaches various methods for applying electrical current to the vertebrae. However, those systems and methods did not envision tailoring and directing the electrical current and electrical field generated by a constant current source to a specific anatomical region of the vertebrae or spine. In the systems described herein, an electric field is generated by selectively energizing one or more conductive components under the desired operating parameters. In the systems and methods used herein, constant current and consistent operating parameters applied to conductive components with varying anodization patterns may produce varying osteogenic and therapeutic results. In some embodiments, varying constant currents and operating parameters may be applied to consistent conductive components to enhance or modify osteogenic and therapeutic results.

As described herein, varied anodization patterns applied to conductive components can be used to selectively enhance osteogenesis in distinct regions of the vertebrae and spine in proximity to the components described herein.

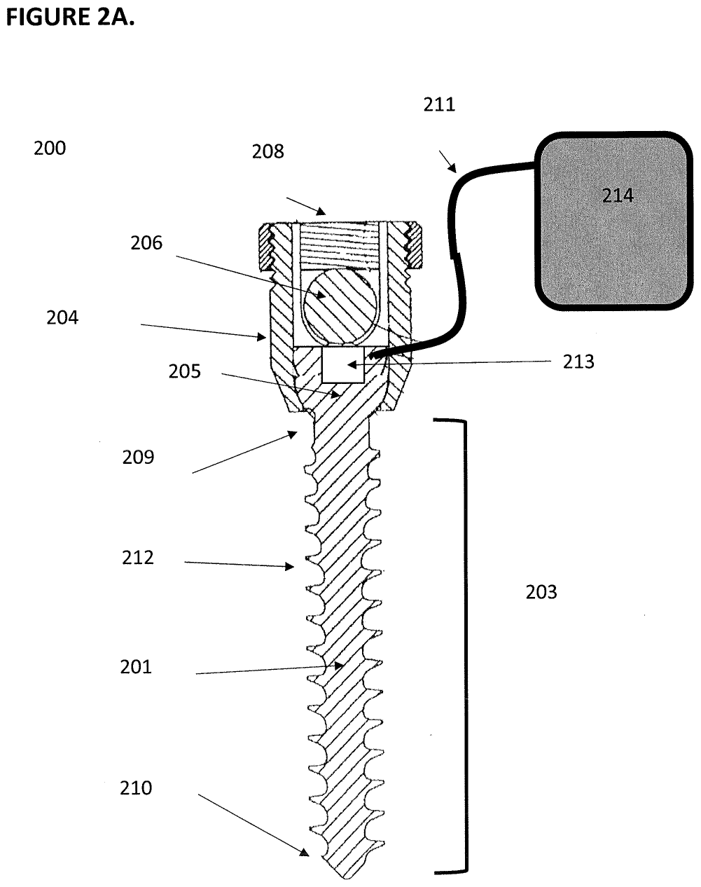

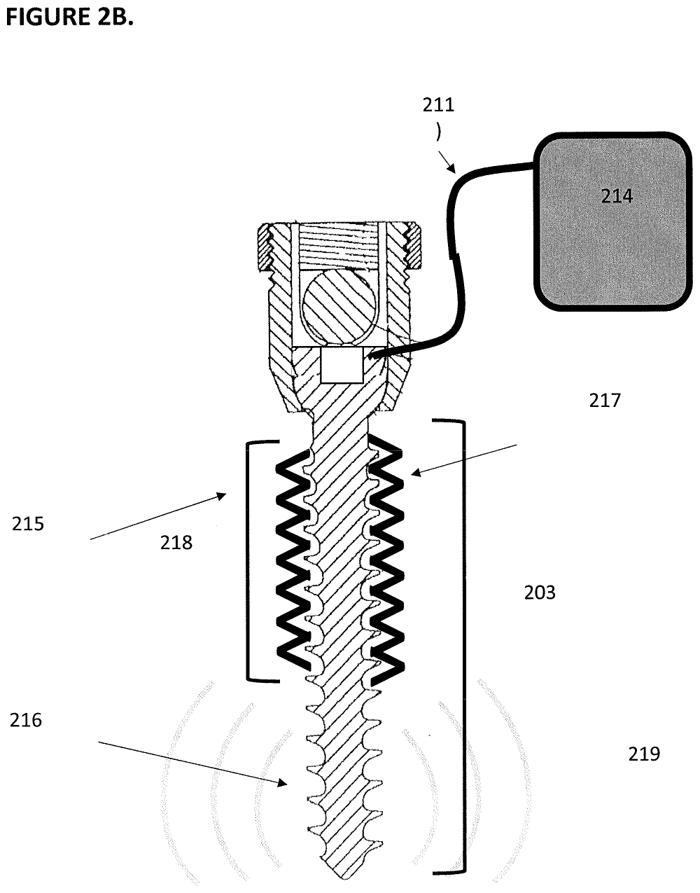

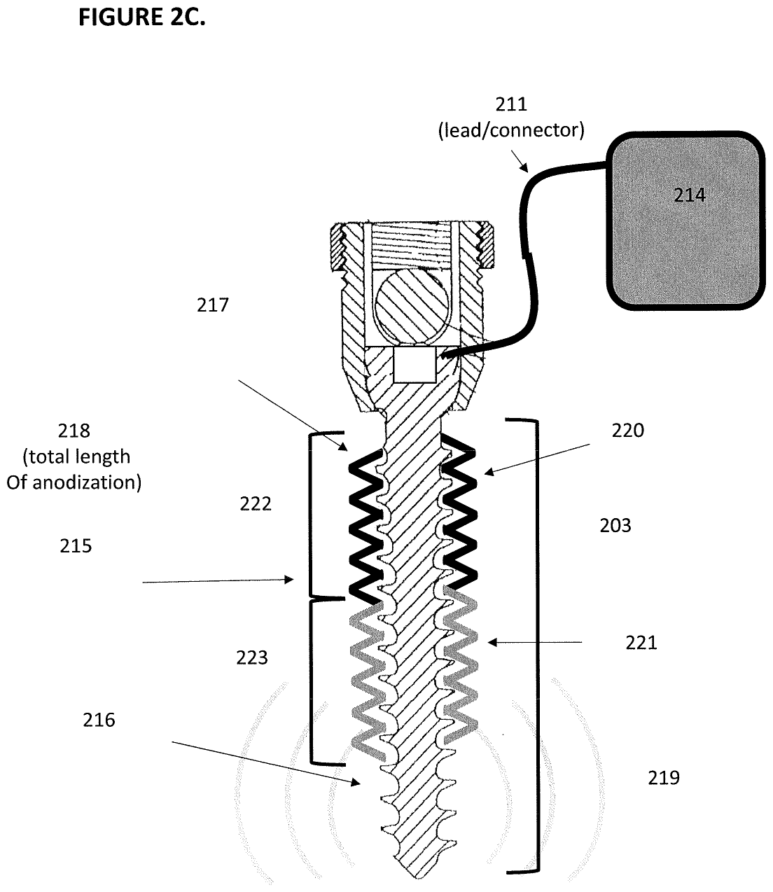

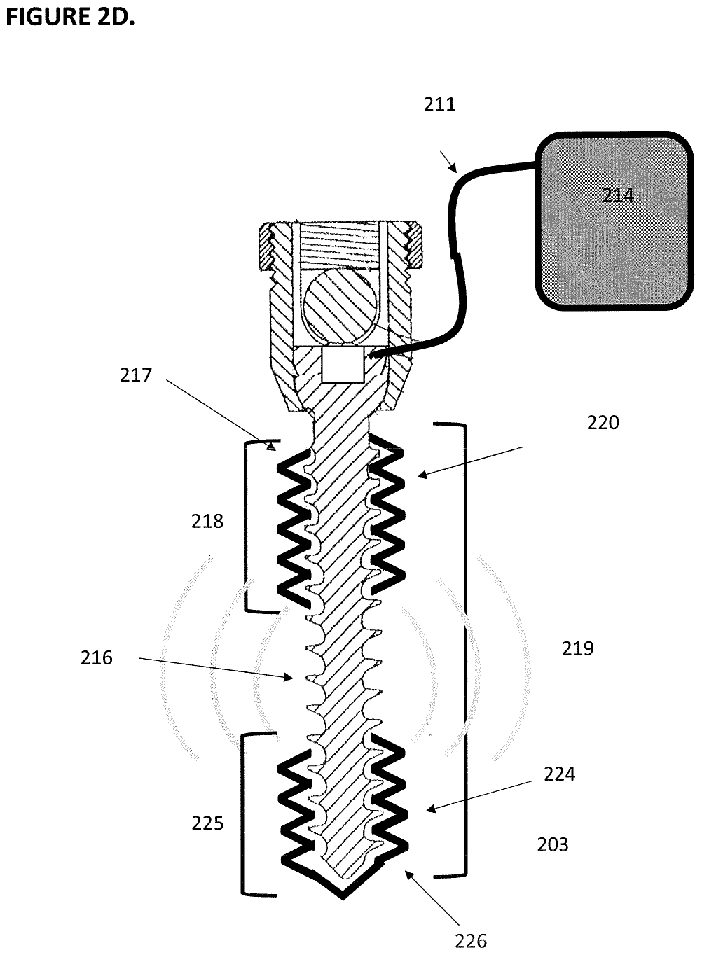

FIGS. 2A-2D illustrate a cross sectional view of various embodiments of fixation systems and conductive components (e.g. screws) and anodization patterns.

FIG. 2A illustrates a cross sectional view of an exemplary fixation system 200 consisting of a conductive screw with anodized surface coating. The fixation system comprises a screw 202, an tulip 204, a rod 206, and a screw cap 208. The screw 200 has an elongated shaft 201 having a length 203 extending between opposite ends 209, 210. A conventional screw thread 212 is formed on an exterior surface of the shaft 201. The thread 212 extends along at least a portion of the length 203 of the shaft 201. The screw 202 also includes a head 205 adjacent the one end 209 of the shaft 201. The head 205 is shaped to include a receptacle 213 (e.g., hex socket) for engaging the screw 200 with a driver or torque wrench to rotate the screw and thereby insert it into bone. In some embodiments, the head 205 and shaft 201 includes an electrical connector, generally designated by 211, for connecting the screw 200 to a power supply or energy source 214, as will be explained in further detail below. As illustrated in FIG. 2A, an electrical conductor 211 is electrically connectable to the screw 202 and to an electrical power source 214 for conveying electrical current through the shaft.

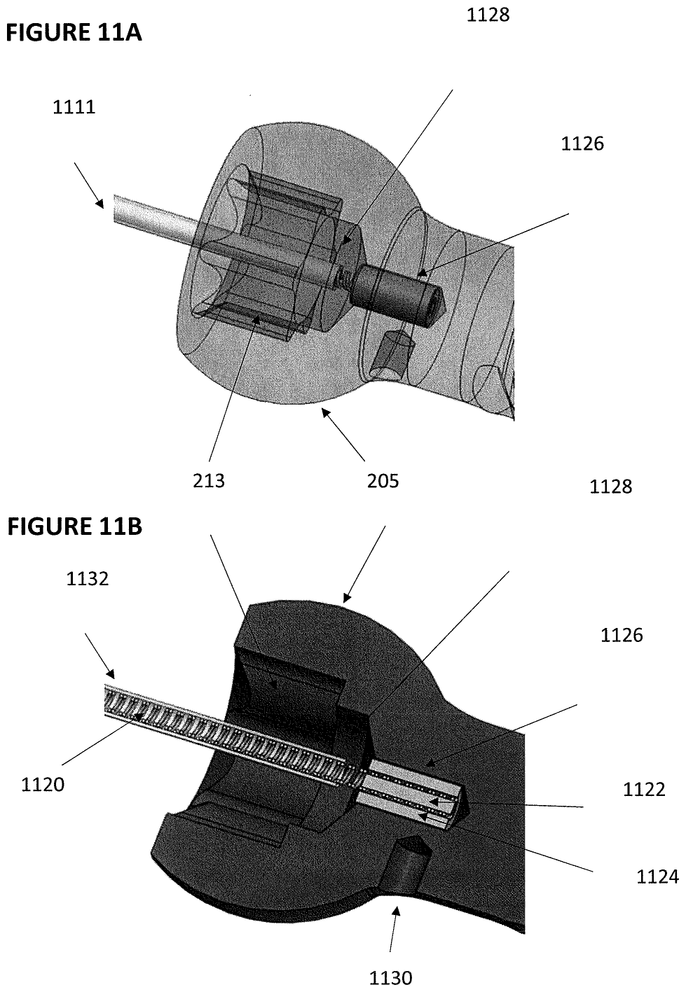

In some embodiments, as shown in FIG. 11A, the connector 1111 includes an multi-conductor electrical lead 1120 (e.g., microwire lead) fitted with a crimp pin 1122 and crimp sleeve 1124 attached to the head 205 in a matching receiving hole 1126 within a vestibule 1128 created at the bottom of the receptacle 213 (e.g., via laser welding) and insulation (e.g., using medical grade epoxy) placed within the vestibule 1128 around the weld. The lead 1120 or connector can comprise an insulator 1132 (e.g., insulating sheath). A feature 1130 forming an insertion point can be positioned at the base of the screw head and can allow welding and/or fixation around the hole 1126 and connector 1111.

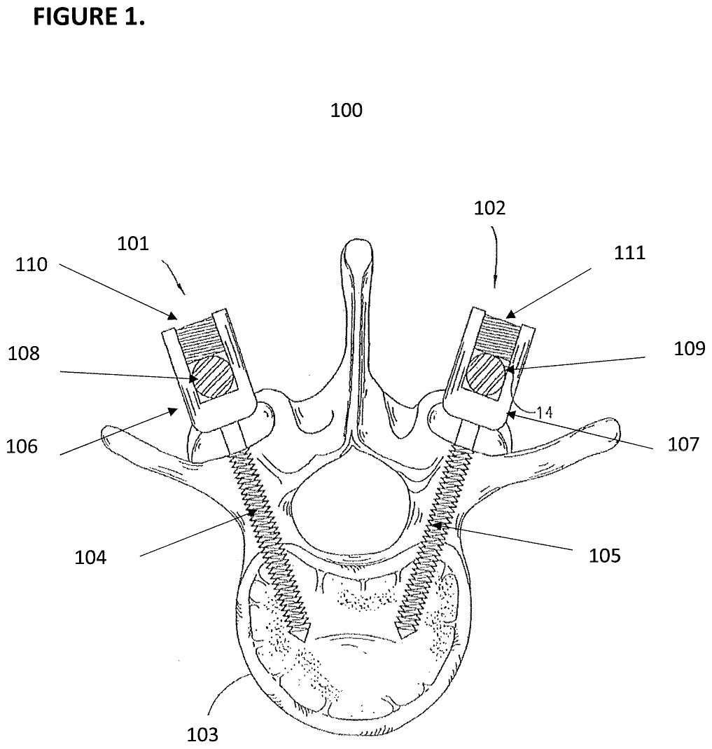

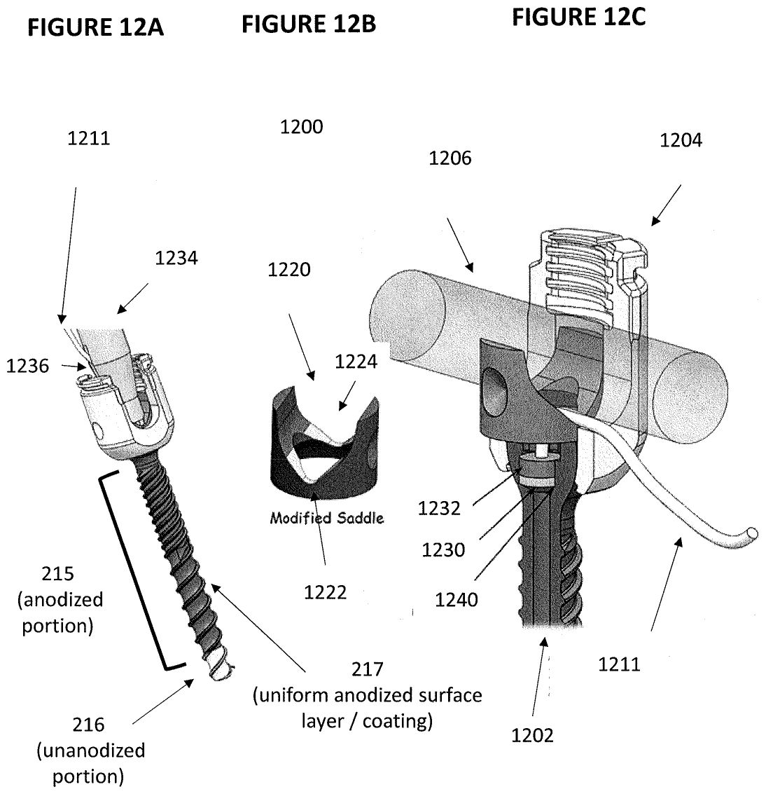

In some embodiments, as shown in FIGS. 12A-12C, the spinal fixation system 1200 comprises a tulip 1204, a rod 1206, a saddle 1220, and a screw 1202. As described above with respect to FIGS. 11A and 1B, and as shown in FIG. 12C, the screw 1202 can comprise a connector attached to a contact 1230 within the screw head. A seal 1232 (e.g., epoxy seal) is provided around the region where the connector 1211 connects to the contact 1230. An insulated portion of the connector 1211 extends from the top of the screw head. The saddle 1220 is configured to sit in the tulip 1204 and comprises a channel 1224 for receiving the rod 1206 at an upper portion of the saddle. A bottom portion of the saddle 1220 is configured to receive at least a portion of the screw 1202 head. The saddle 1220 can help ensure a secure fit between the components of the spinal fixation system 1200, for example, as compared to a system without a saddle 1220. A notch 1222 at a bottom portion of the channel 1224 of the saddle 1220 can allow passage of the connector 1211 from the top of the screw to, for example, a power source or other components without being impinged by the rod 1206. The tulip 1204 may also comprise a notch or a lower side channel to allow passage of the connector 1211.

The system 1200 can be used in combination with driver 1234 that comprises a slot 1236 configured to allow passage of the connector 1211 while engaging the head of the screw 1202. The center of the engaging mechanism of the driver may be open to allow passage of the connector 1211 to the slot 1236.

The top portion of the screw, including the head and a top portion of the shaft (e.g., portion of the shaft without threads) is insulated (e.g., anodized) to prevent current leakage. A small portion 1240 of the screw is left insulated (e.g., masked or anodized removed) to allow passage of current near the contact 1230 portion of the screw. Anodization patterns as described herein may refer to the portion of the screw below the top insulated portion.

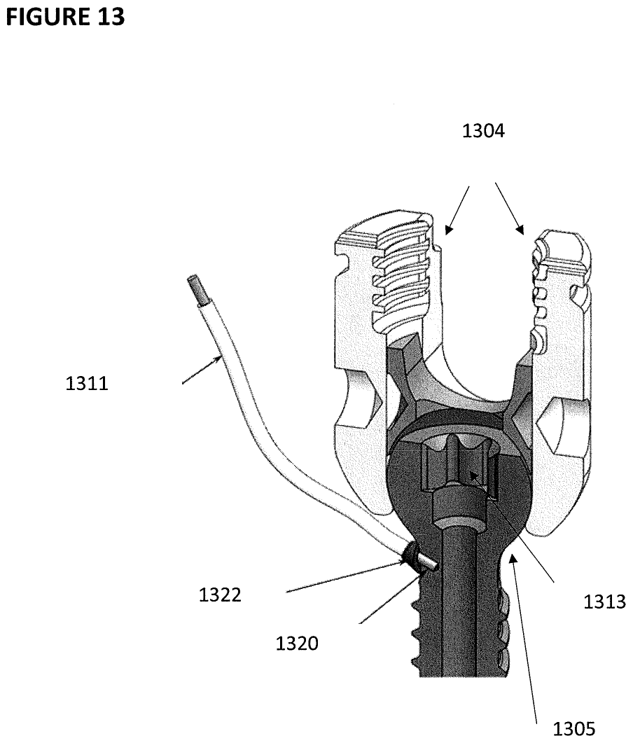

Another embodiment of an electrical connection is shown in FIG. 13. A connector 1311 is inserted at an insertion point 1320 of the shaft of the screw 1302 just below the base of the screw head 1205. An uninsulated tip of the connector 1311 or lead attaches to the screw. The connector 1311 can comprise insulating material (e.g., sealant) 1322 around the insertion point 1320 on an exterior of the screw 1302 to prevent current leakage.

In one embodiment the power source 214 consists of a hermetically sealed titanium enclosure with epoxy header containing one or more medical grade batteries, electrical circuitry, and wireless communication hardware. In this embodiment the power source 214 produces direct current on the order of about 10-100 uA to one or more conductive components (e.g. screws) in an independent and controllable manner. In another embodiment, the power source 214 produces alternating current such as a time-varying current waveform (e.g., a sine wave or a square wave) having a frequency between nearly zero hertz and ten gigahertz.

Although other electrical connectors 211 may be used without departing from the scope of the present invention, in one embodiment the connector 211 is an insulated micro-wire lead consisting of 4 strands of coiled, insulated MP35N microwire encased in a Pellethane sheath. It is further envisioned that the connector 211 may take other forms and may connect to other locations on the screw 202 or component 200. For example, the connector may be a single core microwire or braided microwire attached to the screw 200 at the head 205, within the receptacle 213, on the shaft 201, on the tulip 204, rod 206, or screw cap 208 using a fastenerless connector, clip, soldered pin, or welded pin without departing from the scope of the present invention.

FIG. 2B illustrates a cross sectional view of a fixation system consisting of a conductive screw with single thickness anodized surface coating. The anodized surface coating or layer 217 is applied to the surface of the screw 202 such that and consistent layer of electrically insulating anodization is evenly deposited. The layer of anodization may extend both over the head of the screw 205 and the shaft of the screw 201, evenly coating all surface including the threads 212. In some embodiments the anodization layer 217 consists of Type I anodization created with a driving voltage of >80V. In other embodiments the anodization layer 217 may comprise Type I anodization created with a driving voltage of >60V, >40V, >20V, or >1V. In other embodiments, the anodization layer 217 may comprise Type II, or other types, of anodization. In some embodiments, the layer of anodization 217 extends over the head 205 over a specific length 218 of the screw 202 from one end 209 of the screw toward the opposite end 210 of the screw 202. In some embodiments, the layer of anodization 217 is a consistent thickness/resistivity across the entire anodized length 218 of the screw 202. In some embodiments, the anodized length of the screw 217 extends over 90% of the total length of the screw 203 and the Type I anodized coating (>80V) is a constant thickness creating an even resistive layer of >100 kOhm. The anodized portion 215 of the screw 202 can prohibit delivery of direct current to adjacent bony tissue while the unanodized portion 216 of the screw 202 can support delivery of direct current to adjacent bony tissue. In this manner, electrical currents and electrical fields 219 generated by the attached power supply 214 are specifically directed to the anatomical region of the vertebrae or spine adjacent to the unanodized portion 216 of the screw 202. In some embodiments, a screw 202 with an anodized length 217 extending 90% of the total length of the screw 203 (e.g. about 35 mm) is capable of selectively directing electrical stimulation and fields 219 to the vertebral body and intervertebral disc space (and not the spinal canal) for the purpose of promoting and encouraging interbody fusion. In other embodiments the anodized length 217 may extend about 95%, 50%, 25%, or 0% over the total length of the screw 203. Variation of the anodized length of the screw 217 in these embodiments alters the unanodized length 216 of the screw, thereby changing the anatomical region of the vertebrae or spine adjacent exposed to therapeutic and osteogenic electrical current and fields 219 generated by the power supply 214.

In some embodiments, a screw 202 with an anodized length 217 extending 50% of the total length of the screw 203 is capable of selectively directing electrical stimulation and fields 219 to the vertebral body and pedicles (and not the spinal canal) for the purpose of promoting and encouraging bony fixation and increased screw purchase and retention.

FIG. 2C illustrates a cross sectional view of a fixation system consisting of a conductive screw with varying thickness anodized surface coating. The anodized surface coating or layer 217 is applied to the surface of the screw 202 to create an anodized region 215 utilizing similar methods and types of anodization as described above. The anodized coating is applied such that a variable layer of electrically insulating anodization is deposited along the screw 202. The layer of anodization may extend both over the head of the screw 205 and the shaft of the screw 201, coating all surface including the threads 212. The layer of anodization 217 may also include regions of consistent thickness of anodization 220 extending over a length 222 of the screw 202 and regions of variable thickness of anodization 223 extending of a different length 223 of the screw 202. In some embodiments, the region of anodization 215 may include both layers of constant thickness anodization 220 and variable thickness anodization 221. In these embodiments the length 222 of constant thickness anodization 220 and the length 223 of variable thickness anodization 221 may be varied such that the sum total of the lengths 222, 223 are always less than or equal to the total length of the screw 203. In other embodiments, the region of anodization 215 may only comprise a layer of variable thickness anodization 221 extending over a length 223 of the screw 202. In some embodiments, the thickness of anodization in the variable region 221 may be linearly graded across the anodized length of the screw 223. For example, the thickness of a linearly graded variable thickness region can gradually increase from a thickness of about 0 nm to a thickness of about 500 nm. In other embodiments, the thickness of anodization in the variable region 221 may be exponentially graded across the anodized length of the screw 223. The thickness can exponentially increase from a thickness of about 0 nm to a thickness of about 500 nm. The consistently anodized portion 220 of the screw 202 can completely prohibit delivery of direct current to adjacent bony tissue; the variably anodized portion 221 of the screw 202 can partially prohibit delivery of direct current to adjacent bony tissue; and the unanodized portion 216 of the screw 202 can enable delivery of direct current to adjacent bony tissue. In this manner, electrical currents and electrical fields 219 generated by the attached power supply 214 are specifically directed to the anatomical region of the vertebrae or spine adjacent to the variably anodized 221 and unanodized portion 216 of the screw 202. In some embodiments, a screw 202 comprises an anodized length 217 extending 50% of the total length of the screw 203 and consisting only of a variable layer of anodization 221 linearly graded across the anodized length of the screw 223. The screw 223 can be capable of selectively directing electrical stimulation and fields 219 to the vertebral body and pedicles for the purpose of promoting and encouraging bony fixation and increased screw purchase and retention.

FIG. 2D illustrates a cross sectional view of an embodiment of a fixation system comprising a conductive screw with segmented anodized surface coating. The anodized surface coating or layer 217 is applied to the surface of the screw 202 such that a consistent layer of electrically insulating anodization is evenly deposited in two or more (e.g., 2, 3, 4, 5, or more) discontinuous regions 220, 224 of the screw 202. The anodized surface coating or layer 217 is applied to the surface of the screw 202 to create a first anodized region 220, second anodized region 224, and unanodized region 216 utilizing similar methods and types of anodization as described above. The first region of anodization 220 may extend both over the head of the screw 205 and a length 218 of the shaft of the screw 201, evenly coating all surface including the threads 212. The second region of anodization 224 may extend along a second length 225 of the shaft of the screw 201 and the end of the screw 210, evenly coating all surface including the threads 212 and the tip of the screw. In some embodiments, a region of anodization positioned away from the top end of the screw does not extend all the way to the tip of the screw. In some embodiments, the first region 220 and length 218 of anodization is independent, distinct, and discontinuous from the second region 224 and length 225 of anodization. The first length 218 and second length 225 of the regions of constant thickness anodization 220, 224 may be independently varied to control the length and position of the unanodized region 216 of the screw 202. In these embodiments, the sum total of the first 218 and second 225 lengths of the anodized regions are always less than the length of the screw 203. In some embodiments, the anodization layer 217 in the first 220 and second 224 regions of anodization consists of Type I anodization created with a driving voltage of >80V, creating a resistive layer >100 kOhm. In some embodiments, the anodized regions 220, 224 of the screw 202 prohibit delivery of direct current to adjacent bony tissue while the unanodized portion 216 of the screw 202 supports delivery of direct current to adjacent bony tissue. In this manner, electrical currents and electrical fields 219 generated by the attached power supply 214 are specifically directed to the anatomical region of the vertebrae or spine adjacent to the unanodized portion 216 of the screw 202. In some embodiments, the first region of anodization 220 extends a length 218 of approximately 60% of the total length of the screw 203 (e.g. total length of about 35 mm) from a first end of the screw 209 toward the second end of the screw 210. This first region of anodization 220 is then opposed by an unanodized region of approximately 30% of the total length of the screw 203 (e.g. 35 mm), which is opposed by a second region of anodization 224 extending a length 225 of approximately 10% of the total length of the screw 203 (e.g. total length of about 35 mm) from the second end of the screw 210 toward the first end of the screw 209. In some embodiments, electrical stimulation and fields 219 are selectively directed and delivered to the vertebral body and intervertebral disc space (and not the spinal canal) for the purpose of promoting and encouraging interbody fusion. In other embodiments the anodized regions 220, 224 and lengths 218, 225 may be varied in order to vary the position and length of the unanodized region of the screw 216. For example, in some embodiments, the first region 220 extends a length of about 30%, 40%, 50%, 70%, about 30-50%, 40-6%, 50-70% or 60-80% of the total length of the screw. In some embodiments, the unanodized region is about 10%, 20%, 40%, 50%, 60%, 20-40%, or 30-50% of the length of the screw. In some embodiments, the second region 224 extends a length of about 20%, 30%, 40%, 50%, 5-15%, 10-20%, or 10-30% of the length of the screw. Variation of the anodized lengths of the screw 218, 225 in these embodiments alters the length of the unanodized region 216 of the screw, thereby changing the anatomical region of the vertebrae or spine adjacent exposed to therapeutic and osteogenic electrical current and fields 219 generated by the power supply 214.

In some embodiments, the screw can have a length of about 35 mm. Other lengths are also possible. For example, the screw can have a length of about 5 mm, about 10 mm, about 15 mm, about 20 mm, about 25 mm, about 30 mm, about 35 mm, about 40 mm, about 45 mm, about 50 mm, about 55 mm, about 60 mm, about 65 mm, about 70 mm, about 1-20 mm, about 20-40 mm, about 40-60 mm, about 60-80 mm, etc.

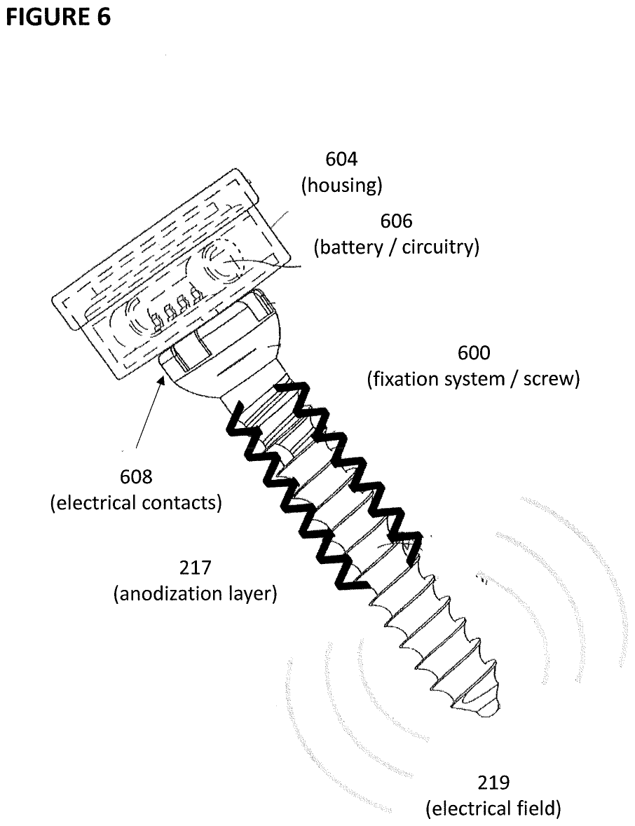

Alternative embodiments of anodization patterns are described in ¶¶ [0040]-[0050] and FIGS. 6-9 of U.S. Publication No. 2014/0200616, filed on Mar. 14, 2014, the entire disclosure of which is herein incorporated by reference.

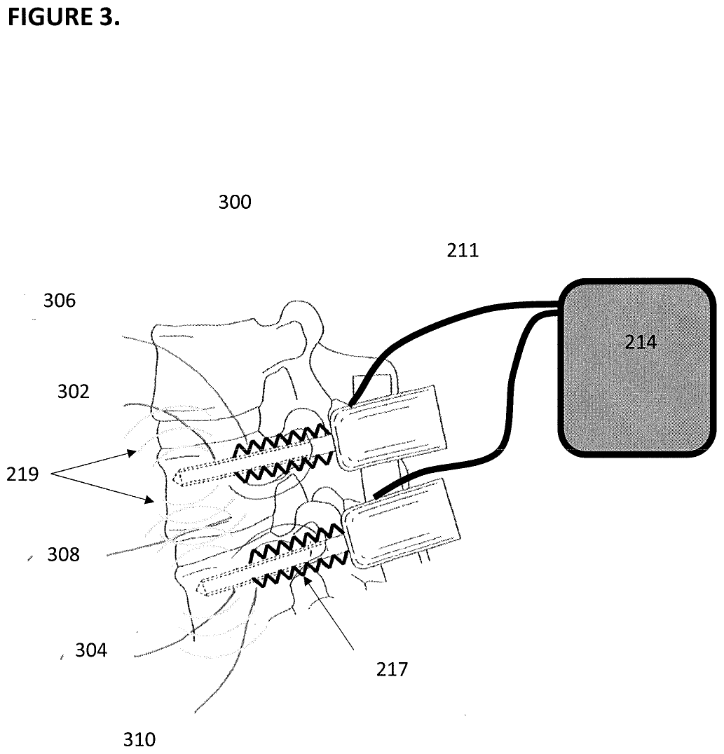

FIG. 3 illustrates an example of a two-level fixation system 300 comprising multiple conductive and selectively anodized screws 302, 304 connected via leads 211 to a power supply 214. The screws 302, 304 possess similar or dissimilar anodization patterns 217 and similar or dissimilar stimulation amplitudes and operating parameters, as described with respect to the embodiments of FIGS. 2A-2D. In some embodiments, the screws 302, 204 possess similar or identical anodization patterns. In these embodiments use of screws with similar anodization patterns may enable uniform and consistent therapeutic and osteogenic stimulation 219 across multiple vertebrae, spinal levels, and sides of the spine. In some embodiments, the screws 302, 304 possess dissimilar anodization patterns. The use of screws with dissimilar anodization patterns may enable distinct and different therapeutic and osteogenic stimulation 219 across specific vertebrae, spinal levels, and sides of the spine. In some embodiments, screws of varying anodization patterns can be selected and utilized according to the anatomical location of the implant, location of the implant within the overall implanted fixation system, the health status/pathological condition of the patient, local bone quality surrounding the implant, and the specific surgical procedure. In some embodiments, the screws 302, 304 work independent of one another to induce distinct and different osteogenic effects 219 only in local tissue directly adjacent to the screws. In some embodiments, the screws 302, 304 work in combination to produce a synergistic electric field 219 that extends either adjacent to or distant from the energized screws. In such embodiments, the electric field 219 in the area 306 distant to screw 302 and in the area 310 distant to screw 304 may be different from the field 219 created in the area 308 in between and in joint proximity to the two screws 302, 304. The field 219 created in the area 306 is a result of the net current supplied from energized screw 302. The field 219 created in the area 308 is a result of the current supplied from energized screw 304. In the interspersed area 308, the field 219 can be a result of the net current supplied both from energized screws 302, 304.

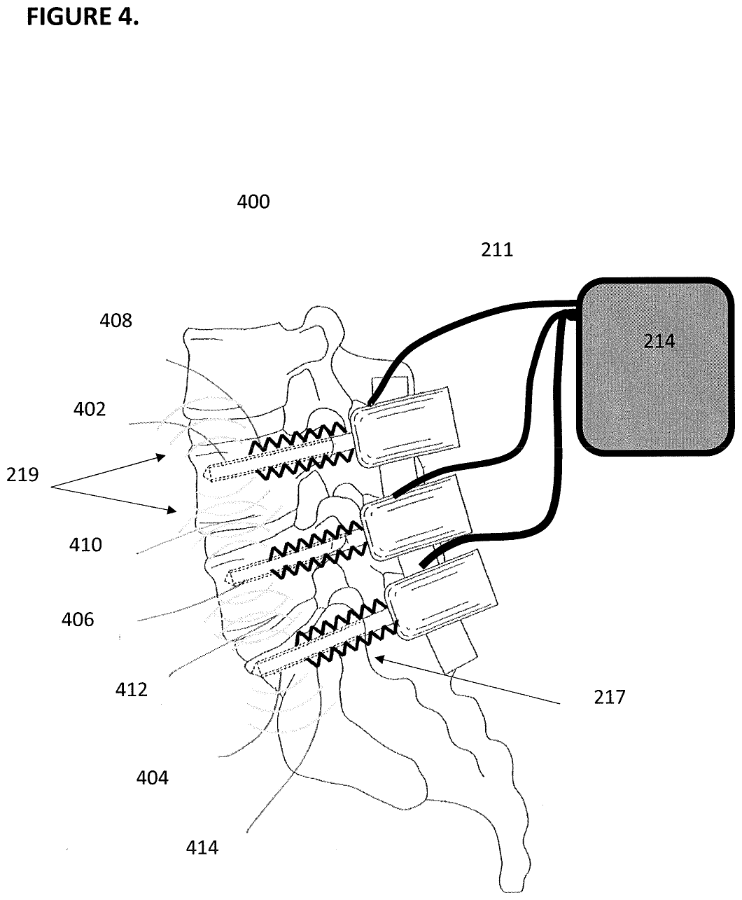

FIG. 4 illustrates an example of a multi-level (e.g. three) level fixation system 400 consisting of multiple conductive and selectively anodized screws 402, 404, 406 connected via leads 211 to a power supply 214. The screws 402, 404, 406 possess similar or dissimilar anodization patterns 217 and similar or dissimilar stimulation amplitudes and operating parameters, as described with respect to the embodiments of FIGS. 2A-2D. In some embodiments, the screws 402, 404, 406 possess similar or identical anodization patterns. The use of screws with similar anodization patterns may enable uniform and consistent therapeutic and osteogenic stimulation 219 across multiple vertebrae, spinal levels, and sides of the spine. In some embodiments, the screws 402, 404, 406 possess dissimilar anodization patterns. The use of screws with dissimilar anodization patterns may enable distinct and different therapeutic and osteogenic stimulation 219 across specific vertebrae, spinal levels, and sides of the spine. In the present fixation system screws of varying anodization patterns can be selected and utilized according to the anatomical location of the implant, location of the implant within the overall implanted fixation system, the health status/pathological condition of the patient, local bone quality surrounding the implant, and the specific surgical procedure. In some embodiments, the screws 402, 404, 406 work independent of one another to induce distinct and different osteogenic effects 219 only in local tissue directly adjacent to the screws. In some embodiments, the screws 402, 404, 406 work in combination to produce a synergistic electric field 219 that extends either adjacent to or distant from the energized screws. In such embodiments, the electric field 219 in the area 208 distant to screw 402 and in the area 414 distant to screw 404 may be different from the fields 219 created in areas 410, 412 in between and in joint proximity to the two screws 402, 406 and 404, 406, respectively. The field 219 created in the area 408 is a result of the net current supplied from energized screw 402. The field 219 created in the area 414 is a result of the current supplied from energized screw 404. In the interspersed areas 410, 412, the fields 219 can be a result of the net current supplied from independent pairs of energized screws 402, 406 and 404, 406, respectively.

The multi-level systems described above can utilize varying screws, anodization, and/or operating parameters at different levels along a same side of the vertebrae or on different sides of the vertebrae. For example, in some embodiments, each level may have screws with a same anodization pattern and opera

In some embodiments of the fixation system the conductive components thereof (e.g. screws) are constructed utilizing medical grade titanium and titanium alloys (e.g. TiAl6V4). Selective surface treatments and layers of anodization may be formed from titanium oxide species (e.g. TiO2). In some embodiments, conductive components of the fixation system may be constructed utilizing other medical grade metallic substrates (e.g. stainless steel, steel alloys, cobalt chrome alloy). In such embodiments, selective surface treatment and electrical insulation may be achieved via application of polymer or adhesive layers. In some embodiments, conductive components of the fixation system may be independently constructed from both medical grade titanium and titanium alloys (e.g. TiAl6V4) and other medical grade metallic substrates (e.g. stainless steel, steel alloys, cobalt chrome alloy). Conductive components of the fixation system may preferably be constructed from low impedance materials in order to adequately route and conduct osteogenic electrical stimuli, while insulting layers of anodized may preferably comprise highly insulating materials in order to prohibit non-specific leakage or release of osteogenic electrical stimuli.

Operating parameters of the system may be controlled and varied to produce varying osteogenic and therapeutic results. In the systems described herein, an electric field is generated by selectively energizing one or more conductive components under the desired operating parameters. In the systems and methods used herein, constant current and consistent operating parameters applied to conductive components with varying anodization patterns may produce varying osteogenic and therapeutic results. In some embodiments, varying constant currents and operating parameters may be applied to consistent conductive components to enhance or modify osteogenic and therapeutic results. In some, an implantable power supply containing an adjustable current-controlled stimulator circuit delivers a constant current (e.g., about 60 μA of direct current) independently to each conductive, selectively anodized pedicle screw via separate micro-wire leads. Integrated sensors and circuitry can continually adjust the compliance voltage according to the measured impedance across each energized screw in order to maintain constant delivery of a constant current (e.g., about 60 μA of direct current) to each screw throughout the treatment period. Other current are also possible (e.g., about 40 μA, about 1-100 μA, about 1-200 μA, about 30-70 μA, about 40-60 μA). In some embodiments, 60 μA of direct current is delivered on a 100% duty cycle for a period of up to 6+months in vivo. Unanodized portions of the conductive screw can serve as independent cathodes in the circuit while the conductive case of the implantable power supply served as the joint anode in the circuit. In some embodiments, the amplitude of electrical stimulation delivered to independent conductive screws may be independently controlled and varied in real-time from 1-100+uA in order to produce varying osteogenic and therapeutic results. For example, increasing current amplitude delivered to a singular conductive screw may focally increase bone formation directly adjacent and proximal to the implanted screw. A high DC current amplitude (80 uA) may be applied to a screw positioned in a compromised/osteoporotic bone/vertebrae in order to enhance the local osteogenic effect and induce more bone growth to compensate for the initial compromised bone quality. A high DC current amplitude (70 uA) may be applied to a screw that is far away from the target region of interest in order to ensure that osteogenic fields are induced in the region despite the increased distance to the target region. A low DC current amplitude (20 uA) may be applied to a screw that is in an area of the bone/vertebrae that does not need as much bone growth/bone formation, or in a sensitive area (e.g., around the spinal canal or foramen) where excessive bone formation may be deleterious. In some embodiments, similar or dissimilar amplitudes of direct current stimulation may be delivered to distinct and independent screws within the fixation system. In some embodiments the duty cycle of direct current electrical stimulation delivered to independent conductive screws may be varied in real-time from 1-100%. A high duty cycle (80%) may be applied to a screw positioned in a compromised/osteoporotic bone/vertebrae in order to enhance the local osteogenic effect and induce more bone growth to compensate for the initial compromised bone quality. A high duty cycle (60%) may be applied to a screw that is far away from the target region of interest in order to ensure that osteogenic fields are induced in the region despite the increased distance to the target region. A low duty cycle (20%) may be applied to a screw that is in an area of the bone/vertebrae that does not need as much bone growth/bone formation, or in a sensitive area (e.g., around the spinal canal or foramen) where excessive bone formation may be deleterious. In some embodiments, similar or dissimilar duty cycles of direct current stimulation may be delivered to distinct and independent screws within the fixation system. For example, increasing the duty cycle of direct current stimulation delivered to a singular conductive screw may focally increase bone formation directly adjacent and proximal to the implanted screw. In some embodiments, similar or dissimilar amplitudes of direct current stimulation may be delivered to distinct and independent screws within the fixation system. In other embodiments the duration of direct current electrical stimulation delivered to independent conductive screws may be varied in real-time from 1 min-6+month, for example, about 30 minutes, about 1 hour, about 6 hours, about 1 day, about 1 week, about 1-3 months, about 2-4 months, about 3-6 months, etc. In some embodiments similar or dissimilar durations of direct current stimulation may be delivered to distinct and independent screws within the fixation system. For example, discontinuation of direct current stimulation delivered to a singular conductive screw may halt bone formation directly adjacent and proximal to the implanted screw if sufficient bone formation or healing has occurred. In contrast, extending the duration of direct current stimulation delivered to a singular conductive screw may promote further bone formation directly adjacent and proximal to the implanted screw if insufficient bone formation or healing has occurred. In another embodiment, alternating current such as a time-varying current waveform (e.g., a sine wave or a square wave) having a frequency between nearly zero hertz and ten gigahertz may be delivered to independent conductive screws.

Alternative embodiments of operating parameters and power sources can be found at ¶¶ [0051]-[0058] of U.S. Publication No. 2014/0200616.

In the present embodiment of the fixation system, electrical stimulation may be applied to conductive components (e.g. screws) by a power supply to induce osteogenic and therapeutic results. The power supply may take multiple implantable forms and may be separate and independent from the conductive hardware of the fixation system, or may be attached to and integrated within the fixation system as later described. In some embodiments, the power source comprises a hermetically sealed titanium enclosure with epoxy header. The titanium enclosure may contain one or more medical grade batteries (e.g. WG9086 batteries), electrical circuitry, microcontrollers, microprocessors, antennas, impedance measurement circuits, and wireless communication hardware. In some embodiments, the power source generates about 10-100 uA in direct current which is independently routed to each energized screw. Each conductive screws can serve as an independent cathode in the circuit while the conductive case of the implantable power supply serves as the joint anode in the circuit. Integrated hardware and circuitry can enable regulation of the amplitude of direct current stimulation on each channel/screw according to the measured impedance across each conductive screw. Additionally, integrated hardware can adjust the compliance voltage through switchable voltage regulators according to measured impedance across each conductive screw. Integrated controllers can allow for on/off control of electrical stimulation applied to each independent screw and for adjustable control of the operating parameters (e.g. current amplitude, duty cycle, duration) of each energized screw. In some embodiments, microcontrollers and microprocessors contained within the power supply facilitate operation of an onboard operating system and real-time data monitoring and recording. Integrated antenna and wireless communication circuitry can enable wireless programming, communication, control, and data-logging with external operating systems, hardware, and software. In some embodiments, a wireless programmer wand connected to a computer running a custom designed software package enables communication with the implanted power supply, programming of the implanted power supply, activation of various operating parameters, and data transmission. In some embodiments, power supplies may incorporate additional sensors, feedback circuits, current modulation circuits, programmable treatment regimens, wireless power/charging modules, and additional advanced electronics common in implantable medical electronics.

In some embodiments, independent feed-throughs are incorporated into the power supply and connected to independent micro-wire leads within an epoxy header. The independent microwire leads can be connected to the head of each conductive screw in order to effectively deliver the generated electrical stimulus to each distinct, addressable screw within the fixation system. Although other electrical connectors may be used without departing from the scope of the present invention, some embodiments utilize insulated micro-wire leads consisting of 4 strands of coiled, insulated MP35N microwire encased in a Pellethane sheath. In other embodiments, electrical connectors may take other forms and may connect to other locations on the screw or on other energized components within the fixation system.

In some embodiments, the spinal fixation system may include unique tools and drivers designed specifically for use with the system and incorporated energized screws. For example, a custom-design driver may be utilized to instrument energized pedicle screws into the bone without impinging or compromising the electrical lead connecting the power supply to the energized screws. A slotted drive shaft can be created to fit and protect the integrated lead during instrumentation, prior to release following screw placement. In some embodiments, custom tools may include tools for attaching, connecting, and anchoring integrated power supplies to the fixation system, and tools for placing and implanting energized components of the fixation system.

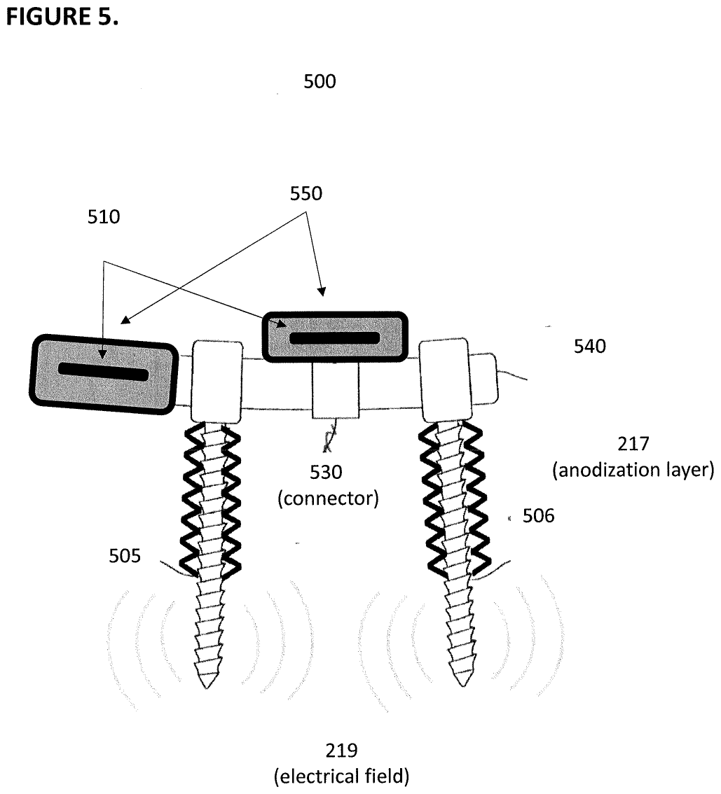

In some embodiments, the spinal fixation system can comprise an attached and integrated power supply rather than a separate and independent power supply. For example, systems such as those described in Sloan (U.S. Patent Publication No. 2015/0088203) can be used. FIG. 5 shows a housing with integrated power supply and attachment device 550 for attachment to a spinal implant system 500 which includes a first pedicle screw 505 and a second pedicle screw 506. In some embodiments of the fixation system, a connector 540 (e.g., a rod) connects the first and second pedicle screws 505, 506 together. The fixation system 500 also contains a housing with integrated power supply 510 adapted for subcutaneous implantation and integration/attachment with the fixation system. In various embodiments, one or more medical grade batteries, electrical circuitry, microcontrollers, microprocessors, antennas, impedance measurement circuits, and wireless communication hardware may be contained within the housing. The device also includes an electrically conductive attachment 550 that attaches and connects the housing 550 to the connector 540 and conducts electrical current from the battery 570 to the connector 540 or rod and thereby to the pedicle screws 505, 506. The housing 550 can be separately and independently attached at multiple positions along the connector 550, and may be multi-plexed such that more than one housing 550 may be attached to a singular connector 540. In some embodiments, the housing 550 may be adapted for attachment to one or more ends of the connector 540 in order to reduce the height and profile of the overall fixation system. The housing 550 may include multiple external buttons or dials for direct manual control of the integrated power supply.

In some embodiments, the spinal fixation system can comprise an integrated power supply located within or directly attached to the screw head rather than a separate and independent power supply. FIG. 6 illustrates a conductive screw 600 comprising an integrated similar to that disclosed in Berger (U.S. Pat. No. 8,380,319). The screw comprises a housing 604 mounted to the head of the screw. In various embodiments, one or more medical grade batteries, electrical circuitry, microcontrollers, microprocessors, antennas, impedance measurement circuits, and wireless communication hardware may be contained within the housing 604. The device also includes an conductive elements 608 that attaches and connects the housing 604 to the screw 600 and conducts electrical current from the battery 606 to the screw 600. The housing 604 can be separately and independently attached at multiple screws 600 within the fixation system. The housing 604 and integrated circuitry may be independently addressable by external communication system in order to control and vary operating parameters for each attached screw 600.

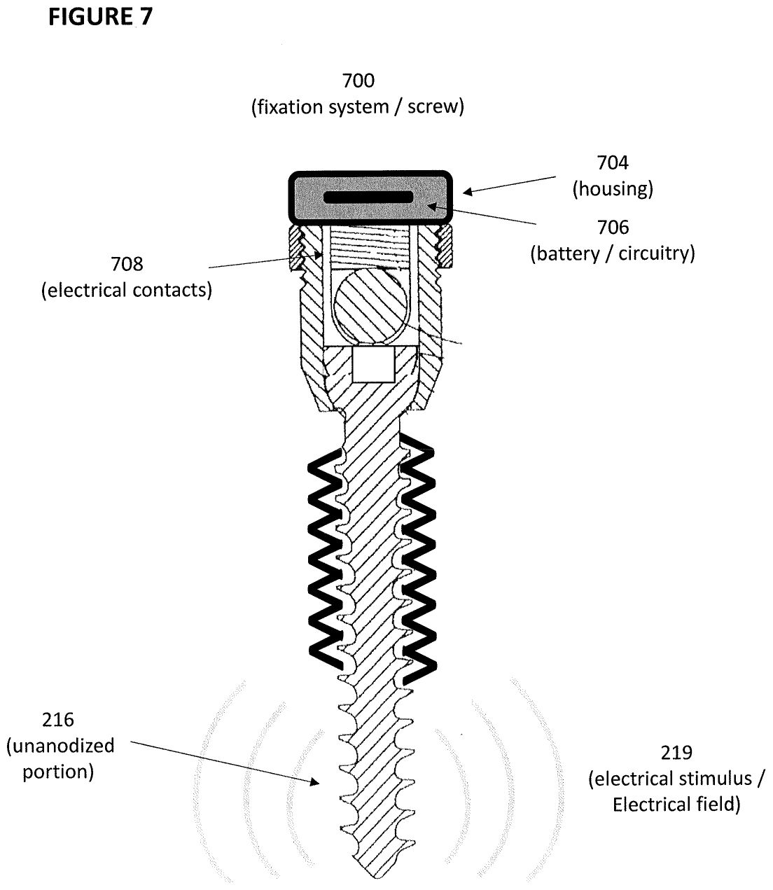

In some embodiments, the spinal fixation system can comprise an integrated power supply located within the screw cap or set screw rather than a separate and independent power supply. FIG. 7 illustrates a conductive screw 700 comprising an integrated housing 704 and battery 706 contained with the screw cap or set screw integrated with the tulip. The conductive and energized screw comprises a housing 704 contained within the screw cap utilized to secure the rod or connector to the tulip and the screw. In various embodiments, one or more medical grade batteries, electrical circuitry, microcontrollers, microprocessors, antennas, impedance measurement circuits, and wireless communication hardware may be contained within the housing 704. The device also includes one or more conductive elements or connectors 708 that make contact with and connect the housing 704 to the electrically conductive tulip and thereby to the screw 600, thereby routing the electrical current from the battery 706 to the screw 700. The housing 704 can be separately and independently attached at multiple screws 700 within the fixation system. The housing 704 and integrated circuitry may be independently addressable by external communication system in order to control and vary operating parameters for each attached screw 700.

In some embodiments, conductive components of the fixation system other than the screw can be utilized to deliver therapeutic electrical stimulation to bony tissues to elicit a desired osteogenic result. For example, in some embodiments, the rod and/or the tulip may be selectively anodized utilizing specific patterns such as those described herein in order to create unanodized regions of the rod and/or the tulip capable of enabling delivery of electrical stimuli to proximal tissues. In some embodiments, the rod within the fixation system may be selectively anodized and energized via an attached or integrated power supply, as described above, in order to focally delivery osteogenic electrical stimulation to the lateral gutter of the spine in one or more locations. Focal delivery of electrical stimulation within the lateral gutter may optimally induce lateral spinal fusion and bone formation. In some embodiments, the tulip within the fixation system may be selectively anodized and energized via an attached or integrated power supply, as described above, in order to focally delivery osteogenic electrical stimulation to the lateral gutter of the spine or the zygopophyseal joints in one or more locations. Focal delivery of electrical stimulation within the lateral gutter may optimally induce lateral spinal fusion and bone formation, while focal delivery of electrical stimulation within the lateral gutter may optimally induce facet fusion.

### Example 1

Instrumented, single-level, posterior lumbar interbody fusion (PLIF) with autologous grant was performed at L4-5 in adult Toggenburg/Alpine goats, using both the spinal systems disclosed herein and standard spinal instrumentation (no electrical stimulation). At terminal time points (3 months, 6 months), animals were killed and lumbar spines were explanted for radiographic analysis using a SOMATOM Dual Source Definition CT Scanner and high-resolution Microcat II CT Scanner. Trabecular continuity, radiodensity, within the fusion mass, and regional bone formation were examined to determine successful spinal fusion.

Osteogenic instrumentation used in the present study consisted of systems described herein configured to focally deliver low-level DC directly into the vertebral bodies including a constant current source, 1 pair of anodized titanium rods, and 2 pairs of selectively anodized pedicle screws. Constant current sources delivering 40 μA DC were a microcircuit board and battery (CR2032 lithium coin cell battery; Varta Microbattery Inc.) sealed in a stainless-steel housing. Titanium rods (5.5 mm diameter, 7.0 cm length), tulips, and screw caps (based on Polaris spinal systems; Biomet Inc.) were custom-milled and hard-anodized to achieve a surface impedance of greater than 1 MΩ, suitable to limit nonspecific current leakage into perispinal tissue. Custom segmental pedicle screws were prepared by selectively anodizing standard segmental pedicle screws (4-mm diameter, 25-mm long; Biomet Inc.). Threaded screw bodies were polished to achieve low surface impedance of less than 5Ω. Selective anodization of pedicle screws enabled selective routing of DC through threaded screw bodies and into the vertebral body. Individual components were assembled intraoperatively to form a complete osteogenic spinal system.

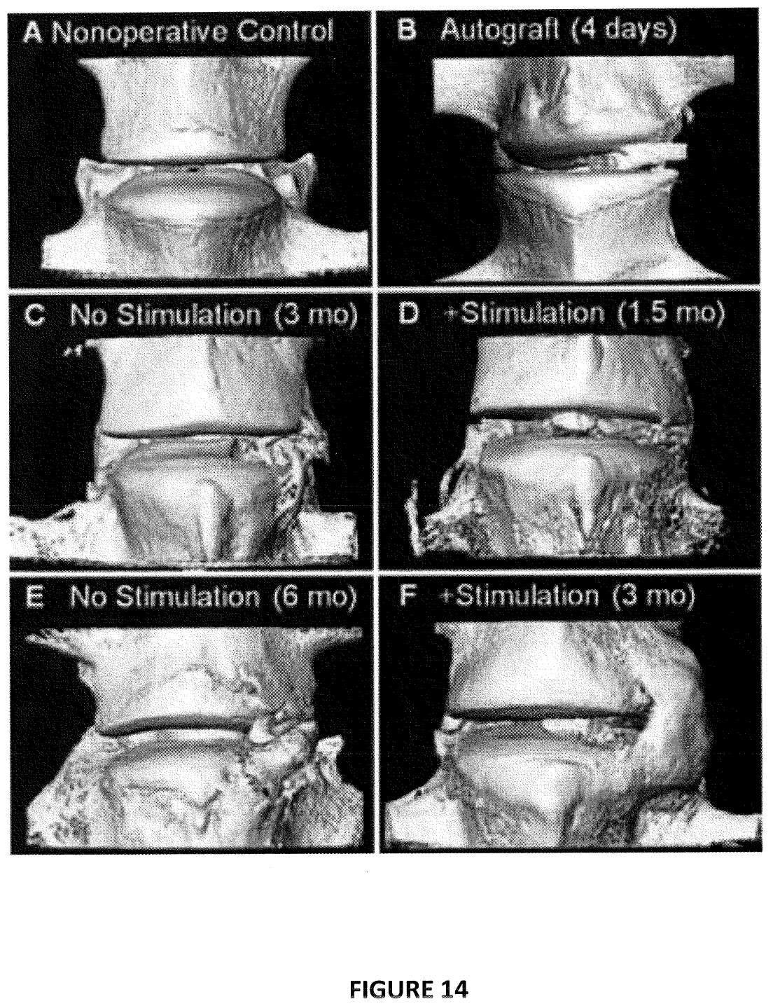

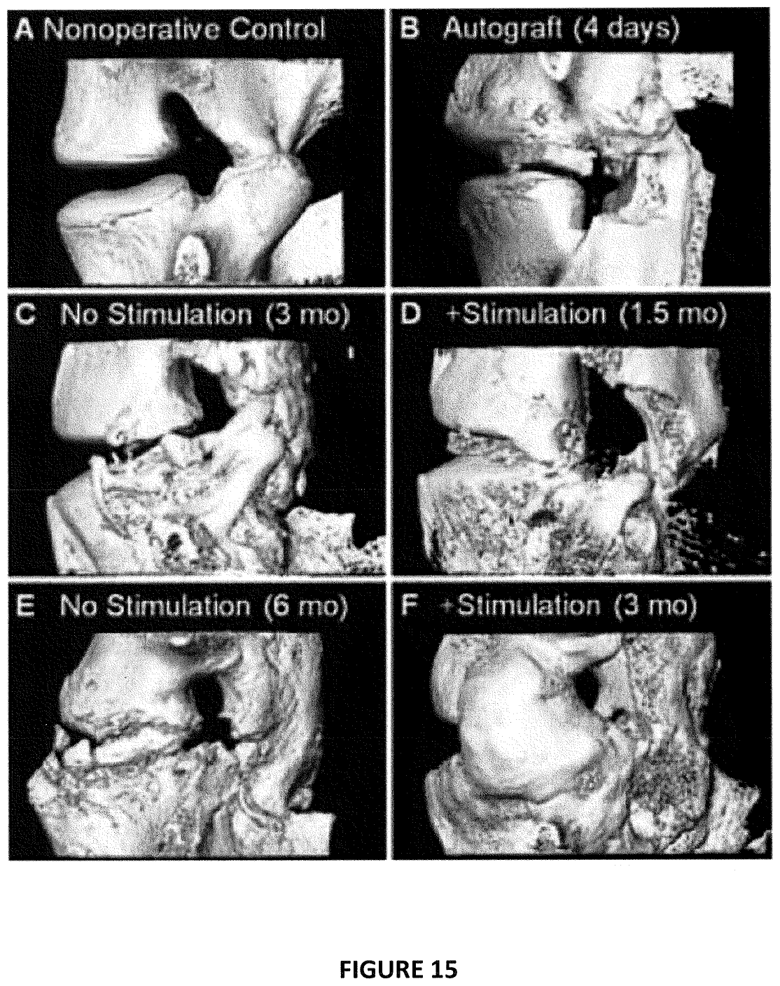

3D reconstruction of micro-CT scans demonstrated increased fusion mass and increased success of fusion in lumbar spines implanted with the osteogenic spinal system. Reconstructions obtained from nonoperative (FIGS. 14A and 15A) and operative disc spaces (FIGS. 14B and 15B) demonstrated successful insertion of autologous bone graft into the L4-5 disc space and negligible anatomical effect of instrumentation at the site of fusion. Reconstructions obtained from spines instrumented with the standard spinal system (no electrical system) for 3 months (FIGS. 14C and 15C) and 6 months (FIGS. 14E and 15E) demonstrated positive signs of bone remodeling yet little fusion mass in the L4-5 disc space and no bony bridging of the vertebral bodies (FIG. 15C, 15E). In contrast, reconstructions obtained from spines instrumented with osteogenic instrumentation (plus electrical stimulation) for 1.5 months (FIGS. 14D and 15D) and 3 months (FIGS. 14F and 15F) demonstrated positive signs of bone remodeling as well as increased fusion mass in the L4-5 disc space and solid bridging of the vertebral bodies.

3D reconstructions further demonstrated increased bone deposition at the site of fusion and increased preservation of autologous bone graft material in the presence of osteogenic spinal instrumentation. Detailed analysis of bony tissue present in the L4-5 disc space revealed a net loss in mineralized bone matrix in the presence of inactive spinal instrumentation (FIG. 14C) and a net gain in mineralized bone matrix in the presence of an osteogenic spinal instrumentation (FIG. 14F). This observation suggests enhanced bone deposition in the presence of electroactive spinal system consistent with prior demonstrations of the osteoinductive effect of DC electrical stimulation. Further examination of 3D reconstructions revealed that autologous bone graft material was better preserved in spines instrumented with osteogenic spinal systems (FIGS. 14D and 15D) than in those with standard systems (FIGS. 14C and 15C). Evidence of bone remodeling and bone depositions in all specimens was focally observed around implanted autologous bone grants.

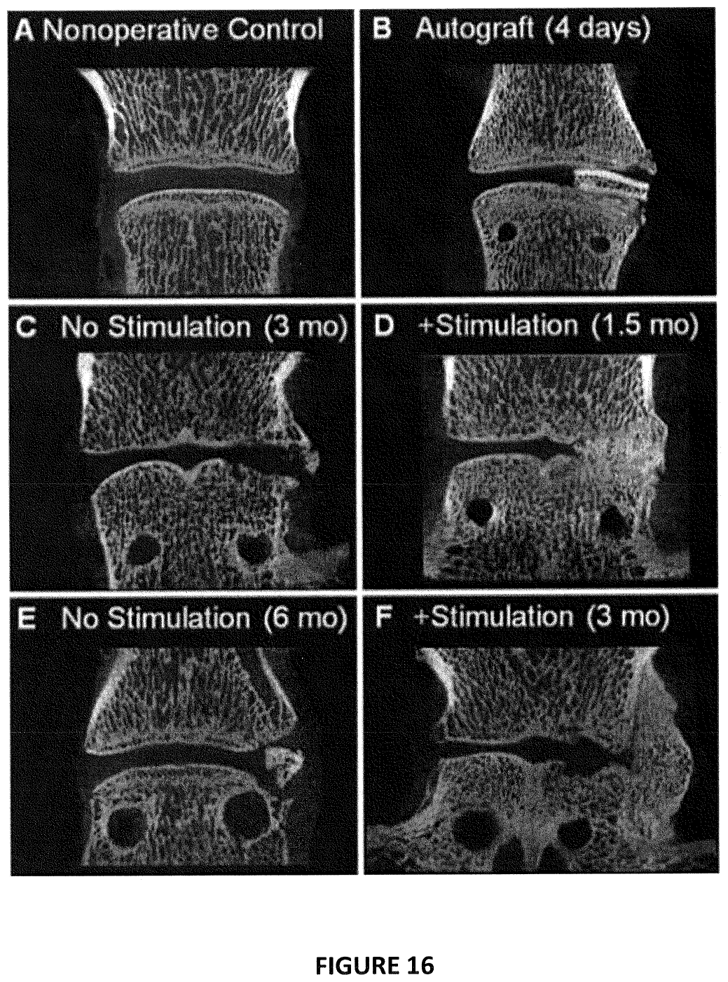

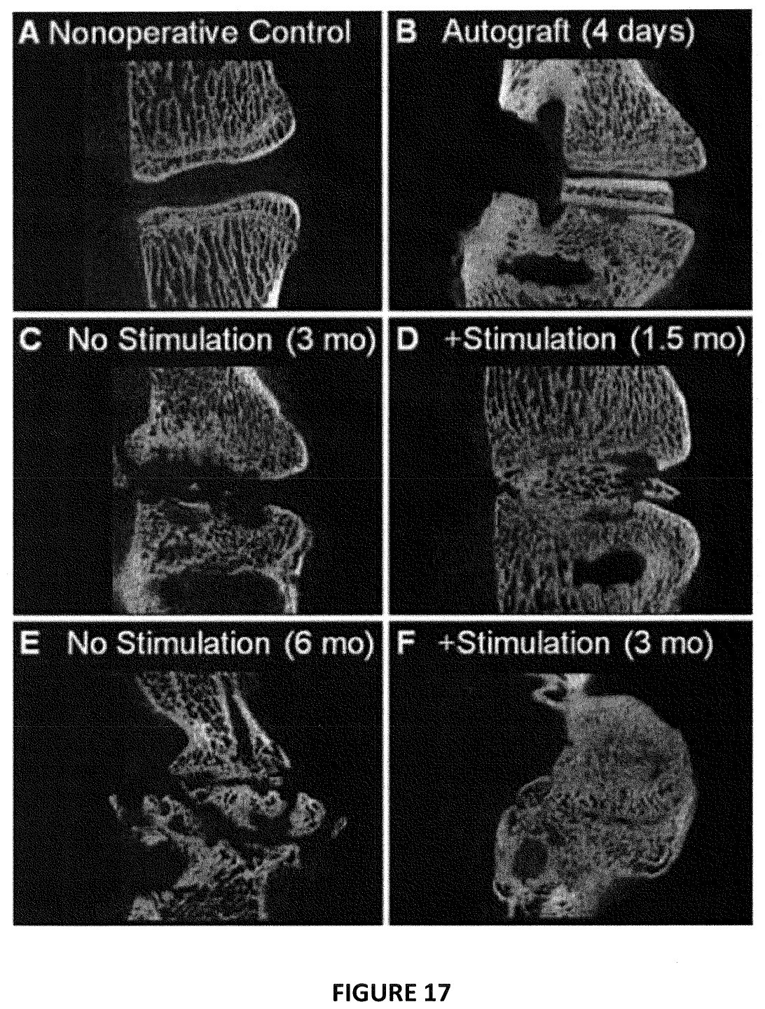

Examination of micro-CT slices confirmed induction of solid bony fusion in the presence of osteogenic spinal instrumentation. Examination of scans obtained from spines instrumented with standard spinal hardware for 3 months (FIGS. 16C and 17C) and 6 months (FIGS. 16E and 17E) demonstrated significant resorption of the bone graft and discontinuity between L-4 and L-5 vertebrae. In contrast, spines instrumented with osteogenic spinal systems for 1.5 months (FIGS. 16D and 17D) and 3 months (FIGS. 16F and 17F) demonstrated mineralized fusion masses within the L4-5 disc space. Despite variability in the mediolateral position of the fusion mass, possibly caused by variation position of implanted bone grafts, all fusion masses successfully bridged L-4 and L-5 vertebrae. Detailed analysis of fusion masses demonstrated continuous regions of woven and trabecular bone, trabecular bridging with vertebral bodies, and consistent radiodensity across the intervertebral disc space.

Micro-CT slices additionally demonstrated local enhancement of bone deposition around implanted osteogenic pedicle screws. Comparison of micro-CT scans obtained from spines instrumented with a standard spinal system for 3 months (FIGS. 16C and 17C) and an osteogenic spinal system for 1.5 months (FIGS. 16D and 17D) revealed increased bone density surrounding pedicle screw beds containing electroactive pedicle screws. Increased bone deposition may result from higher current densities at the bone-screw interface, consistent with prior studies demonstrating a positive correlation between DC density and osteogenic activity.

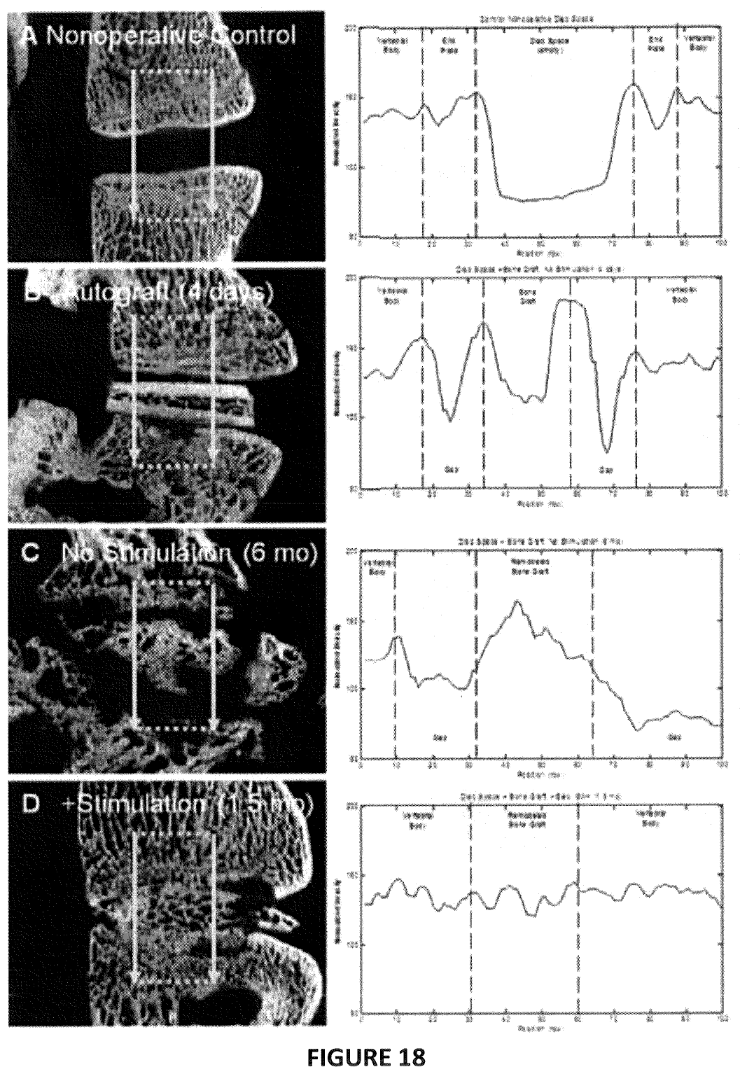

Quantitative analysis of average bone density across the L4-5 disc space confirmed successful interbody fusion in spines instrumented with osteogenic spinal systems. Quantitative analysis demonstrated significant variability in bone density and discontinuity across the L4-5 disc space in spines instrumented with a standard spinal system for 6 months. (FIG. 18C). Evaluation of scans obtained from spines instrumented with an osteogenic spinal system for 1.5 months demonstrated reduced spatial variability in bone density measurements across the target disc space (FIG. 18D). The absence of dramatic minima in bone density plots confirms trabecular continuity between adjacent vertebral bodies and suggests successful graft incorporation in the presence of osteogenic instrumentation.

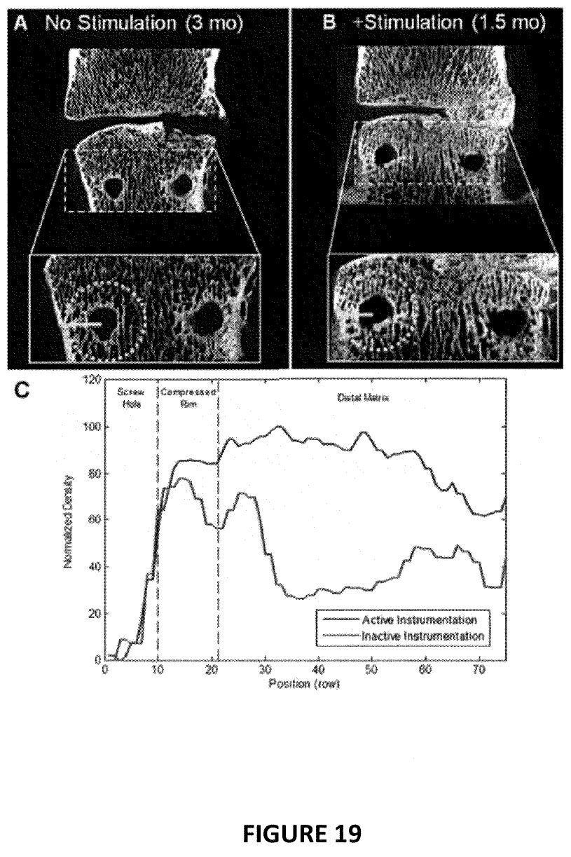

Quantitative analysis of average bone density surrounding pedicle screw beds further confirmed focal enhancement of bone density in the vertebral bodies instrumented with osteogenic pedicle screws. Evaluation of pedicle screw beds in L-5 vertebrae following implantation of standard spinal instrumentation for 3 months (FIG. 19A) and osteogenic spinal instrumentation for 1.5 months (FIG. 19B) demonstrated significant differences in bone density surrounding implanted pedicle screws (FIG. 19C). Both types of pedicle screw induced a high-density rim of compacted bone as a result of instrumentation, yet electroactive pedicle screws were observed to induce higher average bone density beyond the compacted rim compared with standard pedicle screws.

Quantitative analysis of average bone density I pedicle screw beds confirmed that electroactive pedicle screws used in the osteogenic spinal system focally enhanced bone density in instrumented vertebral bodies. Qualitative and quantitative analysis of high-resolution CT scans of explanted lumbar spines further demonstrated that the osteo-genic spinal system induced solid body fusion across the L4-5 disc space as early as 6 weeks postoperatively. In comparison, inactive spinal instrumentation with autograft was unable to promote successful interbody fusion by 6 months postoperatively. These results demonstrate that the osteogenic spinal instrumentation systems disclosed herein support interbody fusion through focal delivery of electrical stimulation.

### Example 2

COMSOL Multiphysics software V4.3 (COMSOL, Inc., Burlington, Mass.) was utilized to simulate the electric field distribution evoked by electroactive pedicle screws in various tissue compartments and anatomical models of the human spine. Electrostatic, AC/DC, and electric current modules were utilized to model the delivery of various amplitudes of DC current from variably anodized pedicle screws. Resulting linear systems of equations were solved using the conjugate gradients solver and plotted in two and three dimensions. Numerical data was exported to MATLAB (MathWorks, Inc., Natick, Mass.) for further data processing and analysis.

Model pedicle screws were based on clinical instrumentation commonly utilized in posteriolateral interbody fusion (PLIF) of the human lumbar spine (screw dia.=6.0 mm, screw length=40 mm). Threaded, high-resolution pedicle screw models were created by importing and rending IGES files of human pedicle screws obtained from GrabCAD, Inc. (Boston, Mass.) in COMSOL. Simplified pedicle screw models, approximated as rounded cylindrical rods, were constructed and rendered in COMSOL. Simplified pedicle screws demonstrated a similar diameter and length to the threaded pedicle screw, yet lacked detailed surface thread patterning. Threaded and simplified pedicle screws were modeled as a single, uniform sub-domain having bulk material properties consistent with medical grade titanium alloy (Ti6Al4V) (σ−2.38 MS/m).

Threaded and simplified pedicle screws were placed in a homogenous, isotropic tissue volume (length=10 cm, width=10 cm, height=10 cm) modeled as a second sub-domain having bulk material properties consistent with either saline (σ=2.0 S/m) or trabecular bone (σ=0.1642 S/m). Electrical activation of model pedicle screws was achieved by assigning current density (Neumann) boundary conditions to the metallic screw sub-domain. Boundary conditions were selected to model DC stimulation amplitudes of 20 uA, 40 uA, 60 uA, 80 uA, and 100 uA. Boundary surfaces of the surrounding tissue volume were set as ground. The electroactive pedicle screw and surrounding tissue volume were discretized into ˜1,000,000 tetrahedrons. Electric field distributions within the tissue volume resulting from electroactive pedicle screws were calculated and plotted in singular colorimetric cross-sections through the tissue volume and the long axis of the screw. Electric field distributions were calculated for various configurations of electroactive pedicle screw, including: variable screw design (threaded/simplified models), and varying stimulation amplitude.

Surface anodization of pedicle screws was modeled as a variable layer of resistive titanium oxide (σ=10 pS/m, thickness=0-400 nm) located at the boundary of the metallic screw sub-domain. Surface anodization was selectively applied to model pedicle screws utilizing either a “uniform paradigm”, in which a uniform anodization thickness (i.e. impedance) is maintained over the anodized region of the screw, or a “graded paradigm”, in which the anodization thickness is graded over the anodized region of the screw. The relative length of the anodized region of pedicle screw to the overall length of the screw was varied between 0%, 50%, 75%, 90%, and 95%. Electric field distributions were calculated for various configurations of the selectively-anodized electroactive pedicle screw, including: variable pattern of anodization (uniform/graded paradigms), and variable length of the anodized region.

A two-level model of the human lumbar spine (L4-L5) instrumented with four electroactive pedicle screws was created to replicate the clinical anatomy following single level posteriolateral interbody fusion (PLIF). High-resolution CAD model of the human spine was obtain from GrabCAD, Inc. (Boston, Mass.) and imported and rendered in COMSOL. L4 and L5 vertebrae were isolated and manually subdivided into cortical and trabecular subdomains with bulk material properties consistent with either cortical (σ=4.52 mS/m transverse, σ=64.52 mS/m horizontal) and trabecular bone (σ=0.1642 S/m transverse, σ=0.2 S/m horizontal). Model selectively-anodized electroactive pedicle screws were inserted into L4 and L5 vertebra using a trans-pedicle approach consistent with current clinical practices. Four total pedicle screws were implemented in the two-level spinal model, with two screws placed into each vertebrae. Two-level vertebral models including instrumented pedicle screws were placed in a homogenous, isotropic tissue volume (length=10 cm, width=10 cm, height=10 cm) modeled as an independent sub-domain having bulk material properties consistent with saline fluid (σ=2.0 S/m). Electrical activation of model instrumented pedicle screws was achieved by assigning electrostatic boundary conditions to the metallic screw sub-domain as previously described. Electric field distributions within the vertebrae and the surrounding tissue volume resulting from activation of instrumented pedicle screws were calculated and plotted in singular colorimetric cross-sections taken at multiple axes through the vertebral model. Electric field distributions were calculated for various configurations of the selectively-anodized electroactive pedicle screw, including: variable pattern of anodization (uniform/graded paradigms), and variable length of the anodized region.

Quantification of induced osteogenic electrical stimuli within key regions of modeled lumbar vertebrae was utilized to compare the potential clinical efficacy of selectively-anodized electroactive pedicle screws. Four regions of interest (ROIs) were identified and defined within the two-level vertebral model: the inter-vertebral disc space (L4-L5 interbody space), vertebral body, pedicle, and spinal canal. Each region was defined as a set of multi-planar two-dimensional surfaces consistent with present anatomical definitions and landmarks. Following calculation of electric field distributions within the vertebrae and the surrounding tissue volume as a result of instrumented electroactive pedicle screws, numerical data obtained from nodes within ROIs was exported and analyzed using MATLAB software.

Electric field distributions within target ROIs were plotted in colorimetric sections taken in the transverse plane through the center of the IV space, L4, and L5 vertebrae and in the saggital plane through the midline of the IV space and spinal canal. Numerical data obtained from nodes within defined ROIs was summed over the surface of the ROI in order to determine a mean value of induced electric field within the anatomical region. Mean electric field amplitude was calculated over both the L4-L5 IV space and the L4-L5 spinal canal and plotted for various configurations of the selectively-anodized electroactive pedicle screw, including: variable pattern of anodization (uniform/graded paradigms), and variable length of the anodized region.

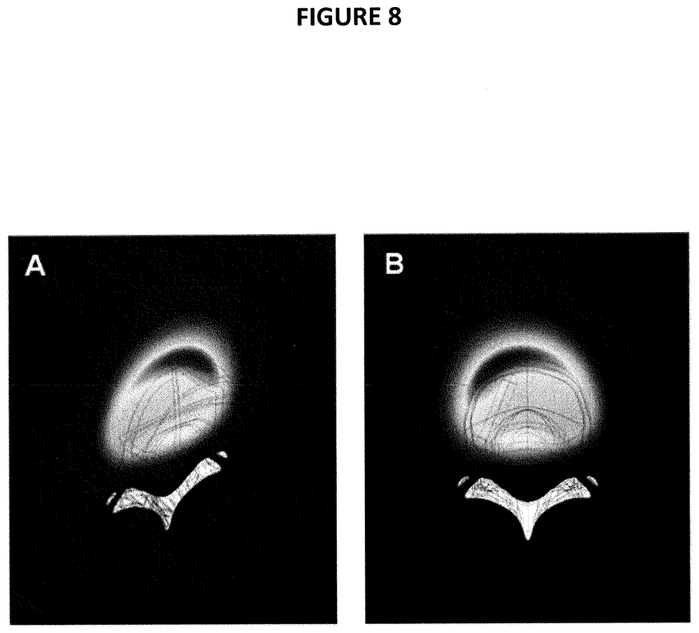

To expand upon the analysis of selective anodization patterns, graded anodization along the length of the pedicle screw body were evaluated in order to determine the effect of non-uniform surface anodization on electric field distributions induced by electroactive pedicle screws. Gradients in the thickness of applied anodization were then applied over the entire length of the screw (100%) or the distal half of the screw (50%). Both linear gradients and exponential gradients were tested. Stimulation amplitude was held constant across all configurations at 40 uA DC. FIG. 8 demonstrates electric field distributions resulting from electrical activation of pedicle screws modified with graded anodization patterns extending over either 100% or 50% of the length of the screw body. Spatial variance of anodization thickness along the length of pedicle screws was observed to further alter the geometry and amplitude of induced electric fields. Pedicle screws modified with a linear gradient of anodization extending from the proximal head of the screw over the entire length of the screw body (100%, linear) exhibited a unique “pear-shaped” electric field distribution (FIG. 8A). The induced electric field at the distal screw tip was higher in amplitude and extended over a larger spatial region, while the field on the proximal portion of the screw body was lower in amplitude and extended only a small distance from the screw surface. Constraining the graded region of anodization to the distal half of the screw (50%, linear) maintained the high amplitude electric field distribution at the distal tip of the screw, while reducing the amplitude and extent of the electric field induced along the proximal portion of the screw (FIG. 8B). Pedicle screws modified with an exponential gradient of anodization extending from the proximal head of the screw over the entire length of the screw body (100%, exponential) exhibited a similar “pear-shaped” electric field geometry with progressively graded features (FIG. 8C). Constraining the graded region of anodization to the distal half of the screw (50%, exponential) resulted in a similar reduction in the proximal field (FIG. 8D).

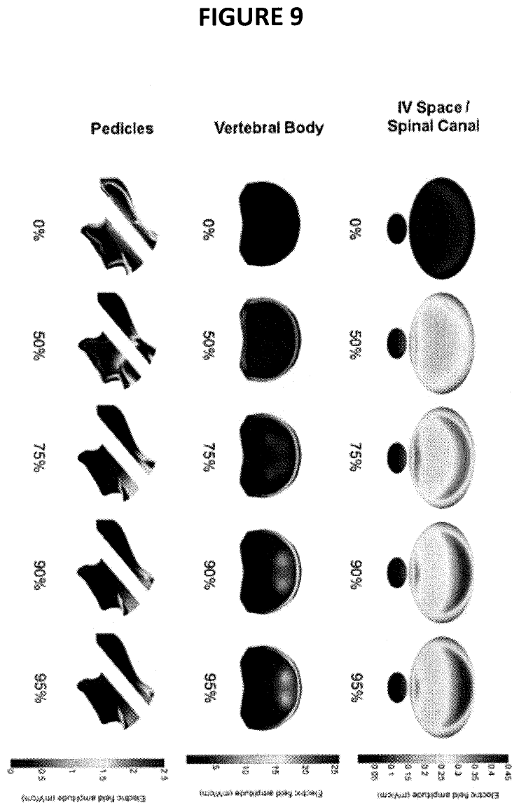

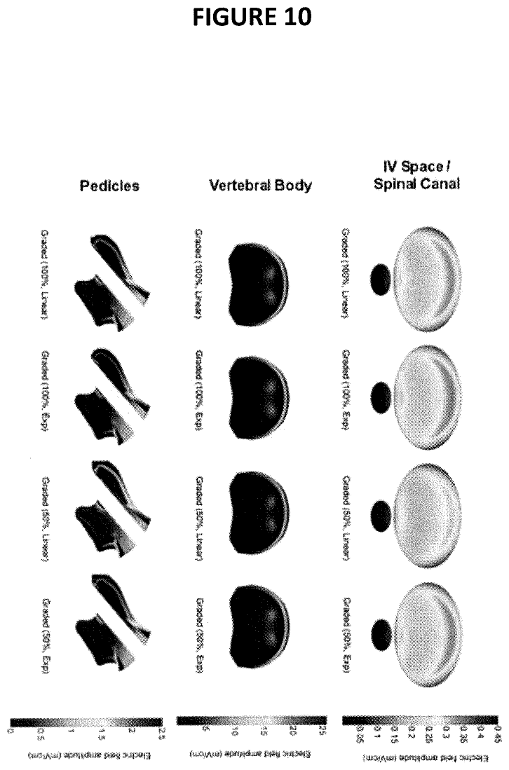

A one-level model of the human lumbar spine (L4-L5) was created to evaluate the capability of instrumented, selectively-anodized pedicle screws to deliver osteogenic electrical stimuli to critical regions of the lumbar spine. illustrates the one-level model of the instrumented lumbar spine constructed to replicate the clinical anatomy following single level posterolateral interbody fusion (PLIF). FIGS. 9B-9E illustrate a high magnification view of the one-level spinal model instrumented with four electroactive simplified pedicle screws from the lateral, superior, posterior, and anterior views, respectively. Electroactive pedicle screws were instrumented into model lumbar vertebrae utilizing angles of approach consistent with present clinical practice. In order to evaluate the ability of electroactive pedicle screw to focally deliver electrical stimuli to clinically-relevant anatomical regions of the lumbar spine four regions of interest (ROIs) were identified: the L4-L5 disc space, vertebral body, pedicle, and spinal canal. FIG. 10 is similar to FIG. 9, but shows the electric fields induced by screws anodized with graded (linear or exponential) patterns of anodization along the length of the screw.

Results obtained from simulations run with the one-level spinal model demonstrated effective induction of therapeutic electric fields within multiple ROIs of the lumbar spine. Un-anodized pedicle screws (0% anodized) induced only low amplitude electric fields within the IV space, spinal canal, and L4 vertebra, and moderate electrical field within the cortical bone of instrumented pedicles. In contrast, anodized pedicle screws (50% anodized) induced high amplitude electric fields within the IV space and L4 vertebra, moderate electrical fields within instrumented pedicles, and low amplitude electric fields in the spinal canal. Comparative analysis of anodization patterns further demonstrate that pedicle screws anodized over 95% of the length of the screw body delivered the greatest amplitude of electrical stimulation to the IV space and L4 vertebral body, and negligible stimulation to the spinal canal. Demonstration of the ability of selectively-anodized pedicle screws to focally induce therapeutic electric fields within the disc space without concomitant induction in the spinal canal confirm the controllable and tunable nature of the osteoinductive system.

Data sets also confirmed the validity of the model as the spatial distribution of induced electric fields within the vertebra and surrounding regions largely matched spatial patterns illustrated in initial uniform tissue volumes. Specifically, induced field amplitudes were noted to be higher in the vertebral body and the pedicle, areas of greater proximity to the screw tip and great conductivity, than in the IV space and the spinal canal, areas of lower proximity to the screw tip and lower conductivity. Similarly, electric fields were notably higher in the cortical bone within the vertebral body than in the trabecular core of the vertebral body.

What is claimed is:

1. A system for spinal fixation and osteogenesis comprising
a pedicle screw comprising a selectively anodized surface configured to generate a desired electric field when energized;
a power source;
an electrical connector connecting the power source and pedicle screw and configured to provide a constant level of direct current to the pedicle screw; and
a saddle configured to receive the pedicle screw and comprising a notch configured to allow passage of the electrical connector from the screw to external components.

2. A system for spinal fixation and osteogenesis comprising
a power source;
a tulip comprising a channel;
a rod configured to be positioned within the channel;
a pedicle screw;
a saddle comprising a notch along a bottom surface shaped to mate with a top of the pedicle screw, the saddle configured to be positioned between the tulip and the rod,
wherein at least one of the tulip, rod, screw seat, and pedicle screw comprises a selectively anodized surface configured to generate a desired electric field when energized using a constant current supplied by the power source, and wherein at least one of the tulip.

3. The system of claim 1 or 2, wherein the power source comprises a hermetically sealed titanium enclosure.

4. The system of claim 3, wherein the enclosure comprises a battery.

5. The system of any of the above claims, wherein the power source is configured to produce direct current of about 10-100 μA.

6. The system of any of the above claims, further comprising a wireless communications module.

7. The system of any of the above claims, further comprising electrical circuitry.

8. The system of any of claims 2-7, further comprising an electrical connector configured to connect the power source to the component comprising the selectively anodized surface.

9. The system of claim 1 or 8, wherein the connector comprises an insulated micro-wire lead.

10. The system of any of claims 2-9, wherein the pedicle screw comprises the selectively anodized surface.

11. The system of claim 1 or 10, wherein the connector is attached to the pedicle screw at a head of the screw and the notch in the saddle permits passage of the connector.

12. The system of claim 1 or 10, wherein the selectively anodized surface comprises a layer positioned at a top portion of the pedicle screw.

13. The system of claim 12, wherein the layer extends over at least a portion of a head and a shaft of the screw.

14. The system of claim 1 or 10, wherein the selectively anodized surface extends over about 90% of a total length of the screw.

15. The system of any of the above claims, wherein the selectively anodized surface comprises an anodized portion and an unanodized portion.

16. The system of claim 15, wherein the anodized portion is configured to prohibit delivery of current to adjacent tissue when the system is implanted.

17. The system of claim 15, wherein the unanodized portion is configured to support delivery of current to adjacent tissue when the system is implanted implanted.

18. The system of any of the above claims, wherein the selectively anodized surface is configured to selectively direct electrical stimulation to the vertebral body and intervertebral disc space without directing electrical stimulation to the spinal canal.

19. The system of any of the above claims, wherein the selectively anodized surface comprises a single thickness.

20. The system of any of the above claims, wherein the selectively anodized surface comprises a variable thickness.

21. The system of claim 20, wherein the selectively anodized surface comprises a linearly graded thickness.

22. The system of claim 20, wherein the selectively anodized surface comprises an exponentially graded thickness.

23. The system of any of the above claims, wherein the selectively anodized surface comprises a first region of a consistent thickness anodization and a second region of a variable thickness anodization.

24. The system of claim 23, wherein the first region comprise about 25% of a length of the component.

25. The system of claim 23, wherein the second region comprises about 75% of a length of the component.

26. The system of any of the above claims, wherein the selectively anodized surface comprises a segmented coating comprising two or more discontinuous regions of anodization.

27. The system of claim 26, wherein a first region of anodization is positioned at a top portion of the screw.

28. The system of claim 26, wherein a second region of anodization is positioned at a bottom portion of the screw.

29. The system of claim 27, wherein the first region comprises about 60% a length of the screw.

30. The system of claim 28, wherein second region comprises about 10% a length of the screw.

31. The system of claim 26, wherein an unanodized region comprising about 30% a length of the screw is positioned between the first region and the second region.

32. The system of any of the above claims, wherein the screw has a length of about 35 mm.

33. The system of any of the above claims, wherein the anodized surface is created with a driving voltage of greater than 80V.

34. The system of any of the above claims, wherein the anodized surface comprises Type I anodization.

35. A spinal fixation system comprising
a first selectively anodized pedicle screw configured to be implanted at a first vertebral level;
a second selectively anodized pedicle screw configured to be implanted at a second vertebral level, different from the first level, wherein the first and second screws are configured to deliver a desired electric field to surrounding tissues and structures when energized; and
a power source configured to deliver constant current to the first and second screws.

36. The system of claim 35, wherein the first and second screws have a same anodization pattern.

37. The system of claim 35, wherein the first and second screws have different anodization patterns.

38. The system of claim 35, wherein the first and second screws are configured to function independent of one another to induce osteogenic effect in tissue directly adjacent to each screw when the screws are energized.

39. The system of claim 35, wherein the first and second screws are configured to work in combination to produce a synergistic electric field when the screws are energized.

40. The system of any of claims 35-39, wherein at least one of the screws comprises an anodized layer positioned at a top portion of the pedicle screw.

41. The system of claim 40, wherein the layer extends over at least a portion of a head and a shaft of the screw.

42. The system of claim 40 or 41, wherein the layer extends over about 90% of a total length of the screw.

43. The system of any of claims 35-42, wherein at least one of the screws comprises an anodized surface comprises a single thickness.

44. The system of any of claims 35-43, wherein at least one of the screws comprises an anodized surface comprises a variable thickness.

45. The system of claim 44, wherein the anodized surface comprises a linearly graded thickness.

46. The system of claim 44, wherein the anodized surface comprises an exponentially graded thickness.

47. The system of any of the claims 1-46, wherein the selectively anodized surface comprises a first region of a consistent thickness anodization and a second region of a variable thickness anodization.

48. The system of claim 47, wherein the first region comprise about 25% of a length of the component.

49. The system of claim 47, wherein the second region comprises about 75% of a length of the component.

50. The system of any of claims 1-40, wherein the selectively anodized surface comprises a segmented coating comprising two or more discontinuous regions of anodization.

51. The system of claim 50, wherein a first region of anodization is positioned at a top portion of the screw.

52. The system of claim 50, wherein a second region of anodization is positioned at a bottom portion of the screw.

53. The system of claim 50, wherein the first region comprises about 60% a length of the screw.

54. The system of claim 50, wherein the second region comprises about 10% a length of the screw.

55. The system of claim 50, wherein an unanodized region comprising about 30% a length of the screw is positioned between a first region and a second region.

56. The system of any of claims 35-55, wherein the screw has a length of about 35 mm.

57. The system of any of claims 35-56, wherein the anodized surface is created with a driving voltage of greater than 80V.

58. The system of any of claims 35-57, wherein the anodized surface comprises Type I anodization.

59. The system of any of claims 35-58, wherein a field created in a region distant to the first screw is different from a field created in a region distant to the second screw.

60. The system of any of claims 35-59, further comprising a third selectively anodized pedicle screw configured to be implanted at a third vertebral level, different from the first and second levels, such that the second pedicle screw is positioned between the first and third pedicle screws.

61. The system of claim 60, wherein the third screw has a same anodization pattern as the first and second screws.

62. The system of claim 60, wherein the third screw has a different anodization pattern from the first and second screws.

63. The system of claim 60, wherein the second and third screws are configured to function independent of one another to induce osteogenic effect in tissue directly adjacent to each screw when the screws are energized.

64. The system of claim 60, wherein the second and third screws are configured to work in combination to produce a synergistic electric field when the screws are energized.

65. A method for inducing osteogenic effect comprising selecting an appropriate anodization pattern for a selectively anodized pedicle screw;
implanting a spinal fixation system comprising the selectively anodized pedicle screw;
energizing the pedicle screw using a constant level of direct current, thereby producing a desired electrical field in an area proximate to the pedicle screw; and
producing an osteogenic effect in surrounding tissue and structures.

66. The method of claim 65, wherein the screw comprises an anodized layer positioned at a top portion of the pedicle screw.

67. The method of claim 66, wherein the layer extends over at least a portion of a head and a shaft of the screw.

68. The method of claim 66 or 67, wherein the layer surface extends over about 90% of a total length of the screw.

69. The method of any of claims 65-68, wherein the screw comprises an anodized surface comprises a single thickness.

70. The method of any of claims 65-69, wherein the screw comprises an anodized surface comprises a variable thickness.

71. The method of claim 70, wherein the anodized surface comprises a linearly graded thickness.

72. The method of claim 70, wherein the anodized surface comprises an exponentially graded thickness.

73. The method of any of the claims 65-72, wherein the screw comprises a first region of a consistent thickness anodization and a second region of a variable thickness anodization.

74. The method of claim 73, wherein the first region comprise about 25% of a length of the component.

75. The method of claim 73, wherein the second region comprises about 75% of a length of the component.

76. The method of any of claims 65-75, wherein the screw comprises a segmented coating comprising two or more discontinuous regions of anodization.

77. The method of claim 76, wherein a first region of anodization is positioned at a top portion of the screw.

78. The method of claim 76, wherein a second region of anodization is positioned at a bottom portion of the screw.

79. The method of claim 76, wherein the first region comprises about 60% a length of the screw.

80. The method of claim 78, wherein the second region comprises about 10% a length of the screw.

81. The method of claim 76, wherein an unanodized region comprising about 30% a length of the screw is positioned between a first region and a second region.

82. The method of any of claims 65-81, wherein energizing the screw comprises applying a direct current of about 60 μA.

83. The method of any of claims 65-82, further comprising connecting the screw to a power source.

84. The method of any of claims 65-82, further comprising implanting a second selectively anodized pedicle screw.

85. The method claim 84, further comprising implanting a third selectively anodized pedicle screw.

86. A system for spinal fixation and osteogenesis comprising
a pedicle screw comprising an electrical connector extending from a head of the screw;
a saddle shaped to receive a head of the pedicle screw and comprising a notch configured to allow passage of the electrical connector therethrough;
a tulip configured shaped to receive the saddle; and
a rod shaped to be positioned above the saddle and within a channel of the tulip.

87. The system of claim 86, wherein the screw comprises a selectively anodized surface configured to generate a desired electric field when energized using a constant current.

88. The system of claim 86, wherein the screw comprises a selectively anodized pattern as described at claims 12-31.

89. The system of claims 86-88, wherein the tulip comprises a notch configured to allow passage of the connector therethrough.

90. The system of claim 86-89, wherein the channel of the tulip exposes the notch of the saddle.

91. The system of claims 86-90, further comprising a driver configured to engage the head of the screw and a slot on a side of the driver to allow passage of the connector therethrough.

92. The system of claims 86-90, wherein the screw head comprises an aperture for receiving the connector.

93. The system of claim 92, wherein the aperture is surrounded by a vestibule.

94. The system of claim 94, wherein the vestibule is filled with a sealant around an attachment point of the connector and the screw.

95. The system of claim 92, wherein the aperture is positioned within a receptacle in the screw head for engaging a driver.

96. The system of claims 86-95, wherein the point at which the connector attaches to the screw is insulated.

97. The system of claims 86-96, wherein a top portion of the screw head and the screw is insulated.

98. The system of claim 97, wherein a portion of the screw at which the screw connects to the connector is uninsulated.

99. A pedicle screw, comprising
a head comprising a receptacle shaped to mate with a driver head;
a connector aperture positioned within the receptacle;
a connector attachment configured for attaching the connector to the connector aperture; and
a vestibule surrounding the connector aperture.

100. The pedicle screw of claim 99, further comprising a connector positioned within the connector aperture.

101. The pedicle screw of claim 100, further comprising sealant positioned within the vestibule and around the connector.

102. The pedicle screw of claim 99, further comprising a channel in a side wall of the pedicle screw allowing access to the vestibule.

103. The pedicle screw of claim 99, further comprising an anodization pattern as described at claims 12-31.
